(19) United States
(12) Patent Application Publication
(10) Pub. No.: US 2026/0285812 A1
(43) Pub. Date: Sep. 24, 2026

(54) QUINOLINEIMINE ANTIDEGRADANT COMPOUNDS AND USES THEREOF

(71) Applicant: FLEXSYS IP HOLDINGS, LLC, Akron, OH (US)

(72) Inventors: Malik Hani AL-AFYOUNI, Akron, OH (US); Judicael Jacques CHAPELET, Copley, OH (US); Donald L. FIELDS, Jr., Copley, OH (US); Frederick IGNATZ-HOOVER, Elyria, OH (US); Jonathan Michael PENNEY, Cuyahoga Falls, OH (US)

(73) Assignee: FLEXSYS IP HOLDINGS, LLC, Akron, OH (US)

(21) Appl. No.: 19/164,044

(22) PCT Filed: Mar. 8, 2024
(86) PCT No.: PCT/US2024/019155

§ 371 Date: Sep. 10, 2025

Related U.S. Application Data
(60) Provisional application No. 63/489,704, filed on Mar. 10, 2023.
(60) Provisional application No. 63/505,909, filed on Jun. 2, 2023.

(51) Int. Cl. C07D 215/38; B29D 30/54; B60C 1/00; C07D 215/50; C07D 221/20; C07D 405/12; C07D 471/04; C08K 5/098; C08K 5/18; C08K 5/3437; C08L 7/00; C08L 9/00; C08L 9/06; C08L 23/22

(52) U.S. Cl. CPC C07D 215/38; B29D 30/542; B60C 1/00; C07D 215/50; C07D 221/20; C07D 405/12; C07D 471/04; C08K 5/098; C08K 5/18; C08K 5/3437; C08L 7/00; C08L 9/00; C08L 9/06; C08L 23/22; C08L 2201/08; C08L 2205/08; C08L 2207/20; C08L 2207/32

(57) ABSTRACT

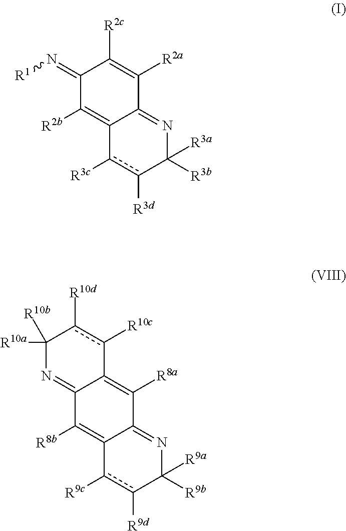

The present disclosure provides compounds represented by Formula (I) or (VIII): (I) (VIII), or salts or solvates thereof, wherein R1, R2a, R2b, R2c, R3a, R3b, R3c, R3d, R8a, R8b, R9a, R9b, R9c, R9d, R10a, R10b, R10c, and R10d are as defined in the specification. The present disclosure also provides compositions, vulcanized elastomeric articles, lubricant compositions, combustible fuel compositions, and fuel additive compositions comprising a compound disclosed herein. The present disclosure also provides processes for preparing the compositions and vulcanized elastomeric articles described herein. The present disclosure also provides a process for retreading tires using a composition described herein. The present disclosure also provides kits comprising a composition described herein.

### BACKGROUND

### Field

The present disclosure provides compounds with antidegradant, e.g., antiozonant, antioxidant and/or antifatigue, properties that are useful additives for vulcanized rubber articles, compositions comprising elastomers, lubricants, fuels, and other compositions which require such properties or in compositions which are themselves useful as compositions to impart such properties.

### Background

Many materials such as plastics, elastomers, elastomeric products (tires, belts, hoses, bushings, mounts, vibration isolators, etc.), lubricants and petroleum products (such as hydraulic fluids, oils, fuels and oil/fuel additives for automotive and aviation applications) are prone to degradation upon prolonged exposure to light, heat, oxygen, ozone, repetitive mechanical actions and the like. Accordingly, compounds and compositions demonstrating antidegradant efficacy are well known in the art. For example, U.S. Pat. No. 8,987,515 discloses an aromatic polyamine useful in inhibiting oxidative degradation particularly in lubricant compositions. U.S. Patent Application Publication No. 2014/0316163 discloses antioxidant macromolecules with purported improved solubility in many commercially available oils and lubricants.

Antidegradants useful in the manufacture of articles formed from elastomers, plastics and the like require a very specific combination of qualities that can be difficult to achieve. While the antidegradants must obviously have commercially acceptable efficacy, they must also exhibit that efficacy over prolonged periods of time associated with use of the article, particularly at exposed surfaces of the article where degradation from environmental factors such as light, oxygen and ozone primarily occurs. Just as important to the protection of surface exposed components, efficacy in protecting imbedded components of composite materials from the effects of oxidative aging and repetitive mechanical action are critically important. The antidegradants must achieve these results while not negatively impacting other additives' efficacy or desirable characteristics in the final article. Further, antidegradants which provide or improve the mechanical fatigue life after an article has been in service, aged oxidatively or by exposure to ozone are highly valued since these will inherently improve the useful mechanical service life of article. Consequently, elastomeric articles which undergo repeated mechanical flexure, extension, or compression during service would greatly benefit from such a discovery.

Articles formed from general purpose elastomers such as natural rubber, in particular tires, are especially prone to degradation from both oxygen and ozone. As discussed in U.S. Pat. No. 2,905,654, the effect on rubber from degradation by oxygen is different from the effect from degradation from ozone; however, both effects can be detrimental to tire performance, appearance and life expectancy. Fatigue and crack propagation are also issues of specific concern, in particular for steel belt edge areas and tire sidewalls which are subject to significant stresses and stretching forces while flexed whether inflated, partially inflated, uninflated and throughout the service life of the tire. U.S. Pat. No. 8,833,417 describes an antioxidant system that purportedly increases longterm resistance to fatigue and crack propagation over the known antioxidants discussed immediately below.

Materials with antidegradant efficacy are well known in the art for use in tire applications and are commercially available. For example, N,N′-disubstituted-paraphenylenediamines such as those sold under the trademark Santoflex™ are generally favored by many tire manufacturers for this purpose. EP 3147321 A1 discloses rubber compositions, tires, amine compounds, and anti-aging agents, and in particular, a rubber composition that is said to be suitable for use in tread rubber or sidewall rubber of a tire.

### BRIEF SUMMARY

The present disclosure provides compounds that are useful as antidegradants, e.g., antiozonants, and/or antioxidants, and/or as additives in lubricants or combustible fuels, represented by any one of Formulae (I)-(X), below, collectively referred to herein as “Compounds of the Disclosure” or individually as a “Compound of the Disclosure.”

The present disclosure also provides compositions comprising:
(i) a Compound of the Disclosure; and
(ii) one or more elastomers; or
(iii) one or more fillers; or
(iv) one or more rubber chemicals; or
(v) one or more plasticizers; or
(vi) one or more additional antidegradants; or
(vii) a combination of one or more elastomers, one or more fillers, one or more rubber chemicals, one or more plasticizers, and/or one or more additional antidegradants, collectively referred to herein as “Compositions of the Disclosure” or individually as a “Composition of the Disclosure.”

The present disclosure also provides Compositions of the Disclosure comprising a Compound of the Disclosure and one or more elastomers.

The present disclosure also provides Compositions of the Disclosure comprising a Compound of the Disclosure and one or more fillers.

The present disclosure also provides Compositions of the Disclosure comprising a Compound of the Disclosure and one or more rubber chemicals.

The present disclosure also provides Compositions of the Disclosure comprising a Compound of the Disclosure and one or more plasticizers.

The present disclosure also provides Compositions of the Disclosure comprising a Compound of the Disclosure and one or more additional antidegradants.

The present disclosure also provides Compositions of the Disclosure comprising a Compound of the Disclosure and one or more carriers.

The present disclosure also provides processes for preparing a composition comprising a Compound of the Disclosure and one or more carriers, the process comprising admixing the compound and the one or more carriers.

The present disclosure also provides vulcanized elastomeric articles comprising a Compound of the Disclosure.

The present disclosure also provides a vehicle comprising a vulcanized elastomeric article, wherein the vulcanized elastomeric article comprises a Compound of the Disclosure.

The present disclosure also provides vulcanized elastomeric articles prepared using a composition described herein, e.g., Compositions of the Disclosure or compositions comprising a Compound of the Disclosure and one or more carriers.

The present disclosure also provides a process for preparing the vulcanized elastomeric articles described herein, the process comprising:
(a) forming a composition described herein into a formed shape; and
(b) vulcanizing the formed shape to provide a vulcanized elastomeric article.

The present disclosure also provides lubricant compositions comprising a lubricant and a Compound of the Disclosure.

The present disclosure also provides combustible fuel compositions comprising a combustible fuel and a Compound of the Disclosure.

The present disclosure also provides fuel additive compositions comprising a fuel additive and a Compound of the Disclosure.

The present disclosure also provides a process for retreading tires, the process comprising:
(a) applying a composition described herein to a tire;
(b) disposing a pre-vulcanized tread around the tire;
(c) disposing a curing envelope around the tire; and
(d) vulcanizing the tire.

The present disclosure also provides kits comprising a composition described herein and instructions for using the composition in a vulcanizable elastomeric composition.

The present disclosure also provides kits comprising a composition described herein and instructions for using the composition to prepare a vulcanized elastomeric article.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1 is an NMR spectrum of Compound 7.

FIG. 2 is a LC chromatogram of Compound 7.

### DETAILED DESCRIPTION

Without wishing to be bound by any particular theory, Applicant has discovered that Compounds of the Disclosure may form bonds with an elastomer during mixing or during the vulcanization process. The vulcanized article thus obtained may comprise both mobile and immobile, i.e., elastomer-bound, compounds having antidegradant properties. It is desirable to find materials which can provide some balance between mobile and immobile protective capacities. For example, in composite articles, mobile additives in formulations are free to migrate between elements of the composite. Meanwhile, allotropes of oxygen can react with component materials of the composite either at the surface of the composite, as does ozone, or anywhere throughout the composite as is the case in diffusion limited oxidation including and especially within internal components of the composite. As a consequence, materials designed to counteract the deleterious effects of oxygen should possess two qualities. To some extent, they should be free to migrate especially to the surface of the composite to protect against the deleterious effects of ozone, but also, so some extent a portion of the material should remain within the component of the composite to which it was originally formulated, thereby providing longterm protection against diffusion limited reactions of oxygen. This is especially desirable for internal components since as surface reactions consume mobile antidegradants, concentrations thereof become depleted within internal components thereby reducing the affordable protection to the internal component. If an antidegradant additive forms a bond to the elastomeric material in a composite, it is ensured to some extent that it will remain within that component throughout the lifetime of that article. Thus, a single additive which can provide protection to the internal components of the composite while at the same time providing protection to the surface of the composite article is highly desirable. This ability to provide some balance of protection to internal components while protecting the surface of the article or composite article is especially desirable in articles expected to have long servicable lifetimes where multiple modes of degradation are possible such as is the case for tires, hoses, belts, bushings, vibration isolators useful for engine mounts, bridge bearings, and or bearings for buildings.

In one embodiment, Compounds of the Disclosure are compounds having Formula (I):

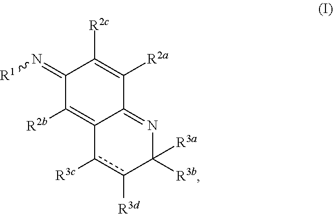

or a salt, solvate, or stereoisomer thereof, wherein:
   [symbol] is a single or a double bond;
   R¹ is selected from the group consisting of optionally substituted C₁-C₁₂ alkyl, —CHR^{1a}R^{1b}, C₃-C₆ cycloalkyl, 4- to 6-membered heterocyclyl, optionally substituted phenyl, and optionally substituted 5- or 6-membered heteroaryl;
   R^{1a} is selected from the group consisting of optionally substituted phenyl, C₃-C₆ cycloalkyl, and optionally substituted 5- or 6-membered heteroaryl;
   R^{1b} is selected from the group consisting of hydrogen and C₁-C₉ alkyl;
   R^{2a}, R^{2b}, and R^{2c} are independently selected from the group consisting of hydrogen, halogen, C₁-C₉ alkyl, C₁-C₉ haloalkyl, C₃-C₈ cycloalkyl, C₁-C₈ alkoxy, C₁-C₈ alkylthio, —C(═O)OR⁴, —OC(═O)R⁴, —C(═O)NR⁴R⁵, —NR⁴C(═O)R⁵, —NR⁴R⁵, —OH, —SH, —SC(═O)R⁴, —SC(═O)SR⁴, —SC(═O)NR⁴R⁵, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino; or
   R^{2a} and R^{2c} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
   R^{2b} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, optionally substituted 4- to 6-membered heterocyclyl, optionally substituted phenyl, optionally substituted 5- or 6-membered heteroaryl, aryloxy, arylthio, and arylamino;
   R^{3a} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and

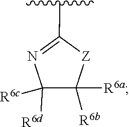

and
R^{3b} is selected from the group consisting of hydrogen, halogen, C₁-C₉ alkyl, and optionally substituted phenyl; or
R^{3a} and R^{3b} taken together with the carbon atom to which they are attached form a C₃-C₁₂ cycloalkyl;
R^{3c} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and

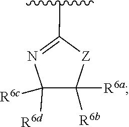

and
R^{3d} is selected from the group consisting of hydrogen, halogen, C₁-C₉ alkyl, and optionally substituted phenyl; or
R^{3c} and R^{3d} taken together with the two carbon atoms to which they are attached form a C₃-C₁₂ cycloalkyl;
R⁴ at each occurrence is independently selected from the group consisting of hydrogen and C₁-C₉ alkyl; and
R⁵ at each occurrence is independently selected from the group consisting of hydrogen and C₁-C₉ alkyl;
R^{6a}, R^{6b}, R^{6c}, and R^{6d} at each occurrence are independently selected from the group consisting of hydrogen, C₁-C₁₂ alkyl, and optionally substituted phenyl;
Z at each occurrence is independently selected from the group consisting of —O—, —S—, and —NR⁷—; and
R⁷ at each occurrence is independently selected from the group consisting of hydrogen and C₁-C₆ alkyl,
with the proviso that the compound of Formula (I) is not:

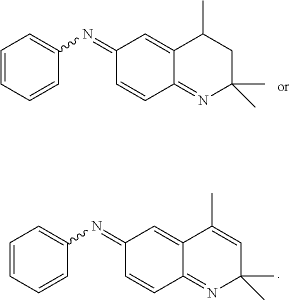 [SMILES: CC1CC(C)(C)N=C2C=CC(=Nc3ccccc3)C=C21 ; CC1=CC(C)(C)N=C2C=CC(=Nc3ccccc3)C=C12]

In some embodiments, Compounds of the Disclosure are compounds having Formula (I), or a salt, solvate, or stereoisomer thereof, wherein:
[symbol] is a single or a double bond;
R¹ is selected from the group consisting of optionally substituted C₁-C₁₂ alkyl, —CHR^{1a}R^{1b}, C₃-C₆ cycloalkyl, 4- to 6-membered heterocyclyl, optionally substituted phenyl, and optionally substituted 5- or 6-membered heteroaryl;
R^{1a} is selected from the group consisting of optionally substituted phenyl, C₃-C₆ cycloalkyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, C₃-C₈ cycloalkyl, C₁-C₈ alkoxy, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino; or
R^{2a} and R^{2c} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{2b} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, optionally substituted 4- to 6-membered heterocyclyl, optionally substituted phenyl, optionally substituted 5- or 6-membered heteroaryl, aryloxy, arylthio, and arylamino;
R^{3a} and R^{3b} are independently selected from the group consisting of hydrogen, halogen, C₁-C₉ alkyl, and optionally substituted phenyl; or
R^{3a} and R^{3b} taken together with the carbon atom to which they are attached form a C₃-C₁₂ cycloalkyl;
R^{3c} and R^{3d} are independently selected from the group consisting of hydrogen, halogen, C₁-C₉ alkyl, and optionally substituted phenyl; or
R^{3c} and R^{3d} taken together with the two carbon atoms to which they are attached form a C₃-C₁₂ cycloalkyl;
R⁴ is selected from the group consisting of hydrogen and C₁-C₉ alkyl; and
R⁵ is selected from the group consisting of hydrogen and C₁-C₉ alkyl,
with the proviso that the compound of Formula (I) is not:

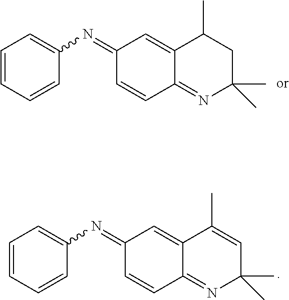 [SMILES: CC1CC(C)(C)N=C2C=CC(=Nc3ccccc3)C=C21 ; CC1=CC(C)(C)N=C2C=CC(=Nc3ccccc3)C=C12]

In some embodiments, Compounds of the Disclosure are compounds having Formula (I), wherein R^{3a}, R^{3b}, and R^{3c} are independently selected from the group consisting of methyl, ethyl, propyl, iso-propyl, n-butyl, sec-butyl, iso-butyl, tert-butyl, pentyl, hexyl, heptyl, octyl, and nonyl.

In some embodiments, Compounds of the Disclosure are compounds having Formula (I), wherein R^{3a}, R^{3b}, and R^{3c} are methyl.

In some embodiments, Compounds of the Disclosure are compounds having Formula (I), wherein R¹ is optionally substituted phenyl.

In some embodiments, Compounds of the Disclosure are compounds having Formula (I), wherein R¹ is substituted phenyl.

In some embodiments, Compounds of the Disclosure are compounds having Formula (I), wherein R¹ is C₁-C₁₂ alkyl.

In another embodiment, Compounds of the Disclosure are compounds having Formula (I), wherein R^{2a}, R^{2b}, and R^{2c} are hydrogen.

In another embodiment, Compounds of the Disclosure are compounds having Formula (I), wherein:
R¹ is selected from the group consisting of optionally substituted C₁-C₁₂ alkyl, —CHR^{1a}R^{1b}, C₃-C₆ cycloalkyl, 4- to 6-membered heterocyclyl, substituted phenyl, and optionally substituted 5- or 6-membered heteroaryl; and
R^{2a}, R^{2b}, and R^{2c} are hydrogen.

In another embodiment, Compounds of the Disclosure are compounds having Formula (I), wherein:
R^{2a}, R^{2b}, and R^{2c} are hydrogen;
R^{3a} is selected from the group consisting of hydrogen, halogen, C₂-C₉ alkyl, and optionally substituted phenyl; and
R^{3b} is selected from the group consisting of hydrogen, halogen, C₁-C₉ alkyl, and optionally substituted phenyl.

In another embodiment, Compounds of the Disclosure are compounds having Formula (I), wherein:
R^{2a}, R^{2b}, and R^{2c} are hydrogen; and
R^{3c} is selected from the group consisting of hydrogen, halogen, C₂-C₉ alkyl, and optionally substituted phenyl; and
R^{3d} is selected from the group consisting of hydrogen, halogen, C₁-C₉ alkyl, and optionally substituted phenyl.

In another embodiment, Compounds of the Disclosure are compounds having Formula (II):

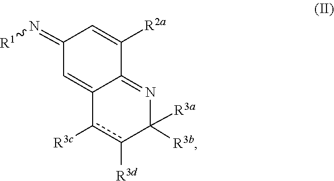

or a salt, solvate, or stereoisomer thereof, wherein
R^{2a} is selected from the group consisting of halogen, C₁-C₉ alkyl, C₁-C₉ haloalkyl, C₃-C₈ cycloalkyl, C₁-C₈ alkoxy, C₁-C₈alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino;
[symbol] is a single or a double bond; and
R¹, R^{3a}, R^{3b}, R^{3c}, R^{3d}, R⁴, and R⁵ are as defined in connection with Formula (I).

In some embodiments, Compounds of the Disclosure are compounds having Formula (II), wherein R^{2a} is C₁-C₉ alkyl.

In some embodiments, Compounds of the Disclosure are compounds having Formula (II), wherein R^{2a} is methyl.

In another embodiment, Compounds of the Disclosure are compounds having Formula (III):

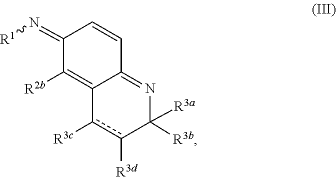

or a salt, solvate, or stereoisomer thereof, wherein
R^{2b} is selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino;
[symbol] is a single or a double bond; and
R¹, R^{3a}, R^{3b}, R^{3c}, R^{3d}, R⁴, and R⁵ are as defined in connection with Formula (I).

In some embodiments, Compounds of the Disclosure are compounds having Formula (III), wherein R^{2b} is C₁-C₉ alkyl.

In some embodiments, Compounds of the Disclosure are compounds having Formula (III), wherein R^{2b} is methyl.

In another embodiment, Compounds of the Disclosure are compounds having Formula (IV):

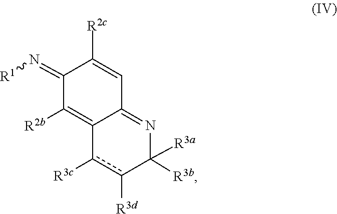

or a salt, solvate, or stereoisomer thereof, wherein:
   R^{2c} is selected from the group consisting of halogen, C₁-C₉ alkyl, C₁-C₉ haloalkyl, C₃-C₈ cycloalkyl, C₁-C₈ alkoxy, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino;
   [symbol] is a single or a double bond; and
   R¹, R^{3a}, R^{3b}, R^{3c}, R^{3d}, R⁴, and R⁵ are as defined in connection with Formula (I).

In some embodiments, Compounds of the Disclosure are compounds having Formula (IV), wherein R^{2c} is C₁-C₉ alkyl.

In some embodiments, Compounds of the Disclosure are compounds having Formula (IV), wherein R^{2c} is methyl.

In another embodiment, Compounds of the Disclosure are compounds having Formula (V):

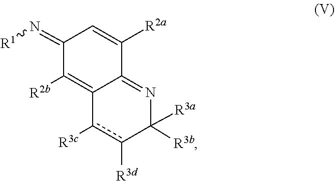

or a salt, solvate, or stereoisomer tereor, wherein:
   R^{2a} and R^{2b} are independently selected from the group consisting of halogen, C₁-C₉ alkyl, C₁-C₉ haloalkyl, C₃-C₈ cycloalkyl, C₁-C₈ alkoxy, C₁-C₈ alkylthio, —C(═O)OR⁴,
   —OC(═O)R⁴, —C(═O)NR⁴R⁵, —NR⁴C(═O)R⁵, —NHR⁴, —NR⁴R⁵, —OH, —SH, —SC(═O)R⁴,
   —SC(═O)SR⁴, —SC(═O)NHR⁴, —SC(═O)NR⁴R⁵, —NHC(═O)SR⁴, —NR⁴C(═O)SR⁵,
   —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino;
   [symbol] is a single or a double bond; and
   R¹, R^{3a}, R^{3b}, R^{3c}, R^{3d}, R⁴, and R⁵ are as defined in connection with Formula (I).

In some embodiments, Compounds of the Disclosure are compounds having Formula (V), wherein R^{2a} and R^{2b} are independently C₁-C₉ alkyl.

In some embodiments, Compounds of the Disclosure are compounds having Formula (V), wherein R^{2a} and R^{2b} are methyl.

In another embodiment, Compounds of the Disclosure are compounds having Formula (VI):

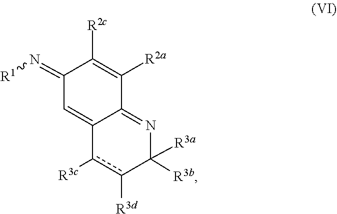

or a salt, solvate, or stereoisomer thereof, wherein:
   R^{2a} and R^{2c} are independently selected from the group consisting of halogen, C₁-C₉ alkyl, C₁-C₉ haloalkyl, C₃-C₈ cycloalkyl, C₁-C₈ alkoxy, C₁-C₈alkylthio, —C(═O)OR⁴, —OC(═O)R⁴, —C(═O)NR⁴R⁵, —NR⁴C(═O)R⁵, —NHR⁴, —NR⁴R⁵, —OH, —SH,
   —SC(═O)R⁴, —SC(═O)SR⁴, —SC(═O)NHR⁴, —SC(═O)NR⁴R⁵, —NHC(═O)SR⁴,
   —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl aryloxy, arylthio, and arylamino;
   [symbol] is a single or a double bond; and
   R¹, R^{3a}, R^{3b}, R^{3c}, R^{3d}, R⁴, and R⁵ are as defined in connection with Formula (I).

In some embodiments, Compounds of the Disclosure are compounds having Formula (VI), wherein R^{2a} and R^{2c} are independently C₁-C₉ alkyl.

In some embodiments, Compounds of the Disclosure are compounds having Formula (VI), wherein R^{2a} and R^{2c} are methyl.

In another embodiment, Compounds of the Disclosure are compounds having Formula (VII):

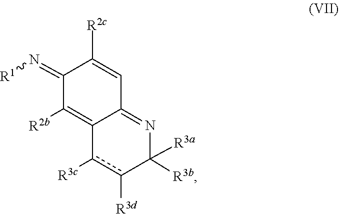

or a salt, solvate, or stereoisomer thereof, wherein:
   R^{2b} and R^{2c} are independently selected from the group consisting of halogen, C₁-C₉ alkyl, C₁-C₉ haloalkyl, C₃-C₈ cycloalkyl, C₁-C₈ alkoxy, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —OH, —SH,
   —SC(═O)R⁴, —SC(═O)SR⁴, —SC(═O)NHR⁴, —SC(═O)NR⁴R⁵, —NHC(═O)SR⁴,
   —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino;
   [symbol] is a single or a double bond; and
   R¹, R^{3a}, R^{3b}, R^{3c}, R^{3d}, R⁴, and R⁵ are as defined in connection with Formula (I).

In some embodiments, Compounds of the Disclosure are compounds having Formula (VII), wherein R^{2b} and R^{2c} are independently C₁-C₉ alkyl.

In some embodiments, Compounds of the Disclosure are compounds having Formula (VII), wherein R^{2b} and R^{2c} are methyl.

In some embodiments, Compounds of the Disclosure are compounds having Formula (I), wherein R^{2a}, R^{2b}, and R^{2c} are independently selected from the group consisting of halogen, C₁-C₉ alkyl, C₁-C₆ alkoxy, and C₁-C₆ alkylthio.

In some embodiments, Compounds of the Disclosure are compounds having any Formulae (II)-(VII), wherein R^{2a}, R^{2b}, and R^{2c} are independently selected from the group consisting of methyl, ethyl, methoxy, chloro, —SCH₃, and —SCH₂CH₃.

In some embodiments, Compounds of the Disclosure are compounds of any one of Formulae (I)-(VII), wherein [symbol] is a double bond.

In some embodiments, Compounds of the Disclosure are compounds of any one of Formulae (I)-(VII), wherein wherein [symbol] is a single bond.

In some embodiments, Compounds of the Disclosure are compounds of any one of Formulae (II)-(VII), wherein R¹ is optionally substituted phenyl.

In some embodiments, Compounds of the Disclosure are compounds of any one of Formulae (II)-(VII), wherein R¹ is C₁-C₁₂ alkyl.

In some embodiments, Compounds of the Disclosure are compounds of any one of Formulae (II)-(VII), wherein R¹ is selected from the group consisting of methyl, ethyl, propyl, iso-propyl, n-butyl, sec-butyl, iso-butyl, tert-butyl, pentyl, hexyl, heptyl, octyl, and nonyl.

In some embodiments, Compounds of the Disclosure are compounds of any one of Formulae (I)-(VII), wherein R¹ is —CHR^{1a}R^{1b}. In some embodiments, R^{1a} is selected from the group consisting of optionally substituted phenyl and optionally substituted 5- or 6-membered heteroaryl. In some embodiments, R^{1a} is phenyl or 2-furyl. In some embodiments, R^{1b} is hydrogen or methyl.

In some embodiments, Compounds of the Disclosure are compounds of any one of Formulae (II)-(VII), wherein R^{3a}, R^{3b}, and R^{3c} are independently selected from the group consisting of methyl, ethyl, propyl, iso-propyl, n-butyl, sec-butyl, iso-butyl, tert-butyl, pentyl, hexyl, heptyl, octyl, and nonyl.

In some embodiments, Compounds of the Disclosure are compounds of any one of Formulae (II)-(VII), wherein R^{3a}, R^{3b}, and R^{3c} are methyl.

In some embodiments, Compounds of the Disclosure are compounds of any one of Formulae (I)-(VII), wherein R^{3b} is methyl; and R^{3a} and R^{3c} are C₂-C₉ alkyl.

In some embodiments, Compounds of the Disclosure are compounds of any one of Formulae (I)-(VII), wherein R^{3b} is methyl; and R^{3a} and R^{3c} are ethyl.

In some embodiments, Compounds of the Disclosure are compounds of any one of Formulae (I)-(VII), wherein R^{3a} is —C(═O)OR⁴. In some embodiments, R^{3a} is —C(═O)OEt.

In some embodiments, Compounds of the Disclosure are compounds of any one of Formulae (I)-(VII), wherein R^{3a} is —C(═O)NR⁴R⁵.

In some embodiments, Compounds of the Disclosure are compounds of any one of Formulae (I)-(VII), wherein R^{3a} is —C(═O)SR⁴.

In some embodiments, Compounds of the Disclosure are compounds of any one of Formulae (I)-(VII), wherein R^{3a} is —CH₂OR⁴.

In some embodiments, Compounds of the Disclosure are compounds of any one of Formulae (I)-(VII), wherein R^{3a} is —CH₂OC(═O)R⁴.

In some embodiments, Compounds of the Disclosure are compounds of any one of Formulae (I)-(VII), wherein R^{3a} is

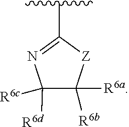

In some embodiments, Compounds of the Disclosure are compounds of any one of Formulae (I)-(VII), wherein R^{3c} is —C(═O)OR⁴. In some embodiments, R^{3c} is —C(═O)OEt.

In some embodiments, Compounds of the Disclosure are compounds of any one of Formulae (I)-(VII), wherein R^{3c} is —C(═O)NR⁴R⁵.

In some embodiments, Compounds of the Disclosure are compounds of any one of Formulae (I)-(VII), wherein R^{3c} is —C(═O)SR⁴.

In some embodiments, Compounds of the Disclosure are compounds of any one of Formulae (I)-(VII), wherein R^{3c} is —CH₂OR⁴.

In some embodiments, Compounds of the Disclosure are compounds of any one of Formulae (I)-(VII), wherein R^{3c} is —CH₂OC(═O)R⁴.

In some embodiments, Compounds of the Disclosure are compounds of any one of Formulae (I)-(VII), wherein R^{3c} is

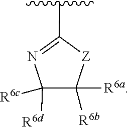

In some embodiments, Compounds of the Disclosure are compounds of any one of Formulae (I)-(VII), wherein Z is —O—. In some embodiments, Z is —S—. In some embodiments, Z is —NR⁷—. In some embodiments, R⁷ is hydrogen. In some embodiments, R^{6a}, R^{6b}, R^{6c}, and R^{6d} are hydrogen.

In some embodiments, Compounds of the Disclosure are compounds of any one of Formulae (I)-(VII), wherein each R⁴ is hydrogen or C₁-C₉ alkyl.

In some embodiments, Compounds of the Disclosure are compounds of any one of Formulae (I)-(VII), wherein each R⁴ is ethyl.

In some embodiments, Compounds of the Disclosure are compounds of any one of Formulae (I)-(VII), wherein each R⁴ is hydrogen.

In some embodiments, Compounds of the Disclosure are compounds of any one of Formulae (I)-(VII), wherein R^{3b} is C₁-C₉ alkyl.

In some embodiments, Compounds of the Disclosure are compounds of any one of Formulae (I)-(VII), wherein R^{3b} is methyl.

In some embodiments, Compounds of the Disclosure are compounds of any one of Formulae (I)-(VII), wherein R^{3b} is methyl; and R^{3a} and R^{3c} are —C(═O)OR⁴.

In some embodiments, Compounds of the Disclosure are any one or more of the compounds of Table 1A, or a salt or solvate thereof.

TABLE 1A

| Cpd | | Cpd | |
|---|---|---|---|
| No. | Structure | No. | |
| 1 | 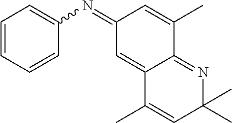 [SMILES: CC1=CC(C)(C)N=C2C(C)=CC(=Nc3ccccc3)C=C12] | 70 | 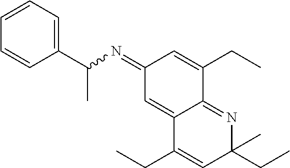 [SMILES: CCC1=CC(C)(CC)N=C2C(CC)=CC(=NC(C)c3ccccc3)C=C12] |
| 2 | 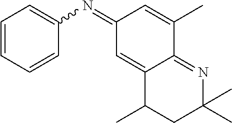 [SMILES: CC1=CC(=Nc2ccccc2)C=C2C1=NC(C)(C)CC2C] | 71 | 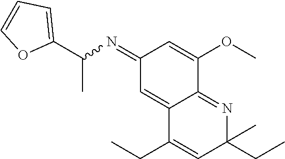 [SMILES: CCC1=CC(C)(CC)N=C2C(OC)=CC(=NC(C)c3ccco3)C=C12] |
| 3 | 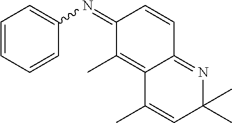 [SMILES: CC1=CC(C)(C)N=C2C=CC(=Nc3ccccc3)C(C)=C12] | 72 | 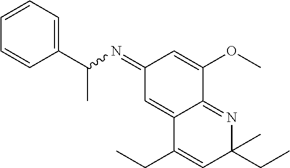 [SMILES: CCC1=CC(C)(CC)N=C2C(OC)=CC(=NC(C)c3ccccc3)C=C12] |
| 4 | 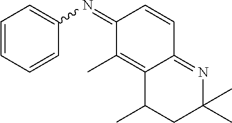 [SMILES: CC1=C2C(=NC(C)(C)CC2C)C=CC1=Nc1ccccc1] | 73 | 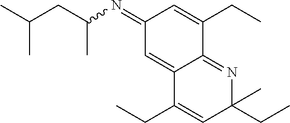 [SMILES: CCC1=CC(C)(CC)N=C2C(CC)=CC(=NC(C)CC(C)C)C=C12] |
| 5 | 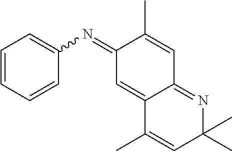 [SMILES: CC1=CC(C)(C)N=C2C=C(C)C(=Nc3ccccc3)C=C12] | 74 | 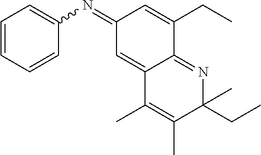 [SMILES: CCC1=CC(=Nc2ccccc2)C=C2C1=NC(C)(CC)C(C)=C2C] |
| 6 | 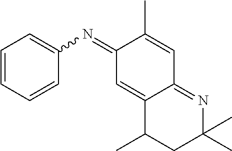 [SMILES: CC1=CC2=NC(C)(C)CC(C)C2=CC1=Nc1ccccc1] | 75 | 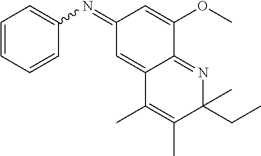 [SMILES: CCC1(C)N=C2C(OC)=CC(=Nc3ccccc3)C=C2C(C)=C1C] |
| 7 | 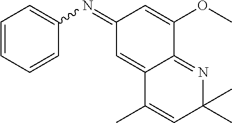 [SMILES: COC1=CC(=Nc2ccccc2)C=C2C(C)=CC(C)(C)N=C12] | 76 | 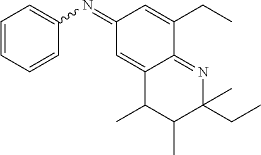 [SMILES: CCC1=CC(=Nc2ccccc2)C=C2C1=NC(C)(CC)C(C)C2C] |
| 8 | 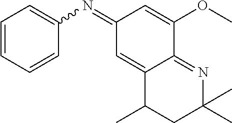 [SMILES: COC1=CC(=Nc2ccccc2)C=C2C1=NC(C)(C)CC2C] | 77 | 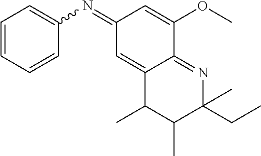 [SMILES: CCC1(C)N=C2C(OC)=CC(=Nc3ccccc3)C=C2C(C)C1C] |
| 9 | 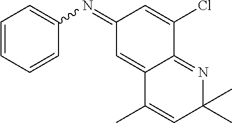 [SMILES: CC1=CC(C)(C)N=C2C(Cl)=CC(=Nc3ccccc3)C=C12] | 78 | 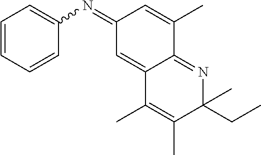 [SMILES: CCC1(C)N=C2C(C)=CC(=Nc3ccccc3)C=C2C(C)=C1C] |
| 10 | 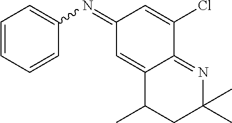 [SMILES: CC1CC(C)(C)N=C2C(Cl)=CC(=Nc3ccccc3)C=C21] | 79 | 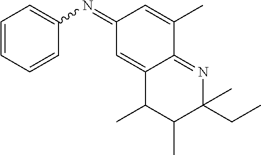 [SMILES: CCC1(C)N=C2C(C)=CC(=Nc3ccccc3)C=C2C(C)C1C] |
| 11 | 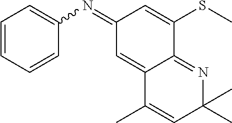 [SMILES: CSC1=CC(=Nc2ccccc2)C=C2C(C)=CC(C)(C)N=C12] | 80 | 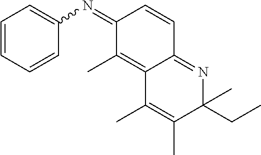 [SMILES: CCC1(C)N=C2C=CC(=Nc3ccccc3)C(C)=C2C(C)=C1C] |
| 12 | 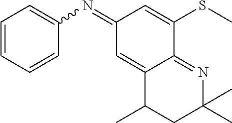 [SMILES: CSC1=CC(=Nc2ccccc2)C=C2C1=NC(C)(C)CC2C] | 81 | 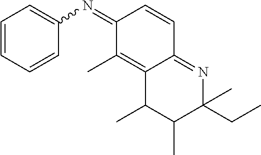 [SMILES: CCC1(C)N=C2C=CC(=Nc3ccccc3)C(C)=C2C(C)C1C] |
| 13 | 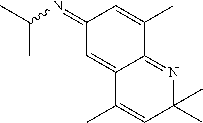 [SMILES: CC1=CC(C)(C)N=C2C(C)=CC(=NC(C)C)C=C12] | 82 | 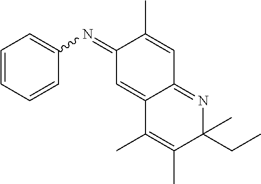 [SMILES: CCC1(C)N=C2C=C(C)C(=Nc3ccccc3)C=C2C(C)=C1C] |
| 14 | 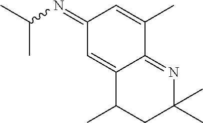 [SMILES: CC1=CC(=NC(C)C)C=C2C1=NC(C)(C)CC2C] | 83 | 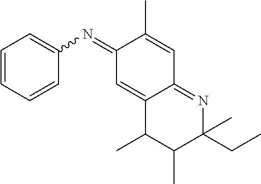 [SMILES: CCC1(C)N=C2C=C(C)C(=Nc3ccccc3)C=C2C(C)C1C] |
| 15 | 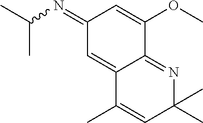 [SMILES: COC1=CC(=NC(C)C)C=C2C(C)=CC(C)(C)N=C12] | 84 | 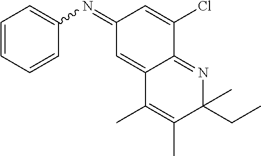 [SMILES: CCC1(C)N=C2C(Cl)=CC(=Nc3ccccc3)C=C2C(C)=C1C] |
| 16 | 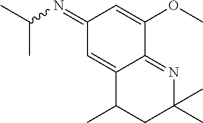 [SMILES: COC1=CC(=NC(C)C)C=C2C1=NC(C)(C)CC2C] | 85 | 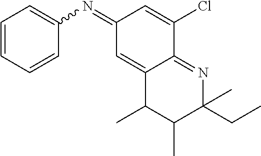 [SMILES: CCC1(C)N=C2C(Cl)=CC(=Nc3ccccc3)C=C2C(C)C1C] |
| 17 | 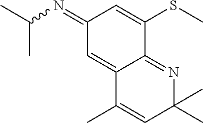 [SMILES: CSC1=CC(=NC(C)C)C=C2C(C)=CC(C)(C)N=C12] | 86 | 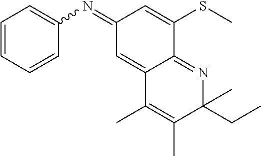 [SMILES: CCC1(C)N=C2C(SC)=CC(=Nc3ccccc3)C=C2C(C)=C1C] |
| 18 | 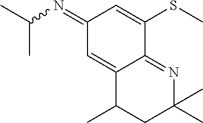 [SMILES: CSC1=CC(=NC(C)C)C=C2C1=NC(C)(C)CC2C] | 87 | 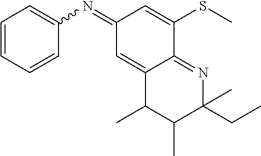 [SMILES: CCC1(C)N=C2C(SC)=CC(=Nc3ccccc3)C=C2C(C)C1C] |
| 19 | 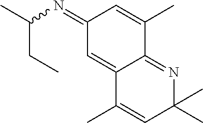 [SMILES: CCC(C)N=C1C=C(C)C2=NC(C)(C)C=C(C)C2=C1] | 88 | 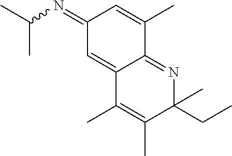 [SMILES: CCC1(C)N=C2C(C)=CC(=NC(C)C)C=C2C(C)=C1C] |
| 20 | 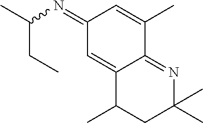 [SMILES: CCC(C)N=C1C=C(C)C2=NC(C)(C)CC(C)C2=C1] | 89 | 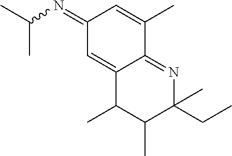 [SMILES: CCC1(C)N=C2C(C)=CC(=NC(C)C)C=C2C(C)C1C] |
| 21 | 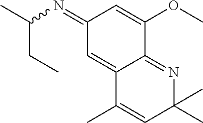 [SMILES: CCC(C)N=C1C=C(OC)C2=NC(C)(C)C=C(C)C2=C1] | 90 | 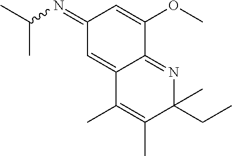 [SMILES: CCC1(C)N=C2C(OC)=CC(=NC(C)C)C=C2C(C)=C1C] |
| 22 | 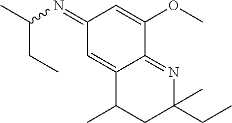 [SMILES: CCC(C)N=C1C=C(OC)C2=NC(C)(CC)CC(C)C2=C1] | 91 | 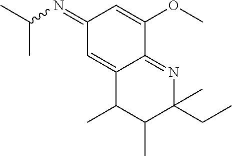 [SMILES: CCC1(C)N=C2C(OC)=CC(=NC(C)C)C=C2C(C)C1C] |
| 23 | 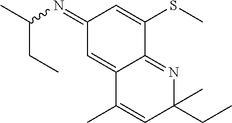 [SMILES: CCC(C)N=C1C=C(SC)C2=NC(C)(CC)C=C(C)C2=C1] | 92 | 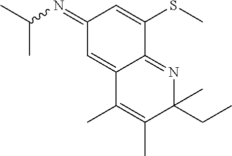 [SMILES: CCC1(C)N=C2C(SC)=CC(=NC(C)C)C=C2C(C)=C1C] |
| 24 | 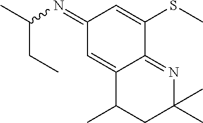 [SMILES: CCC(C)N=C1C=C(SC)C2=NC(C)(C)CC(C)C2=C1] | 93 | 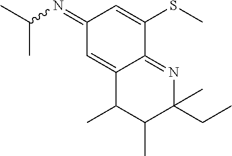 [SMILES: CCC1(C)N=C2C(SC)=CC(=NC(C)C)C=C2C(C)C1C] |
| 25 | 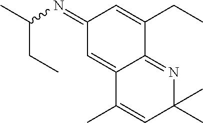 [SMILES: CCC1=CC(=NC(C)CC)C=C2C(C)=CC(C)(C)N=C12] | 94 | 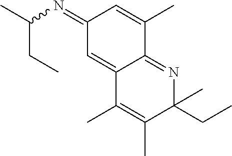 [SMILES: CCC(C)N=C1C=C(C)C2=NC(C)(CC)C(C)=C(C)C2=C1] |
| 26 | 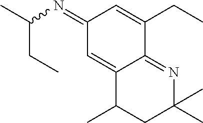 [SMILES: CCC1=CC(=NC(C)CC)C=C2C1=NC(C)(C)CC2C] | 95 | 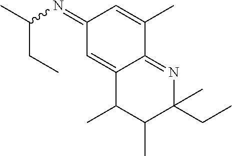 [SMILES: CCC(C)N=C1C=C(C)C2=NC(C)(CC)C(C)C(C)C2=C1] |
| 27 | 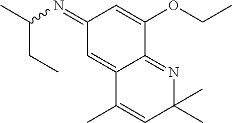 [SMILES: CCOC1=CC(=NC(C)CC)C=C2C(C)=CC(C)(C)N=C12] | 96 | 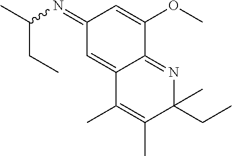 [SMILES: CCC(C)N=C1C=C(OC)C2=NC(C)(CC)C(C)=C(C)C2=C1] |
| 28 | 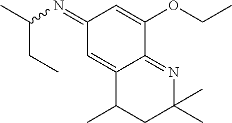 [SMILES: CCOC1=CC(=NC(C)CC)C=C2C1=NC(C)(C)CC2C] | 97 | 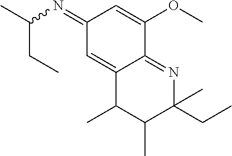 [SMILES: CCC(C)N=C1C=C(OC)C2=NC(C)(CC)C(C)C(C)C2=C1] |
| 29 | 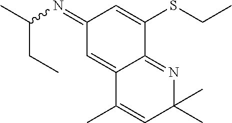 [SMILES: CCSC1=CC(=NC(C)CC)C=C2C(C)=CC(C)(C)N=C12] | 98 | 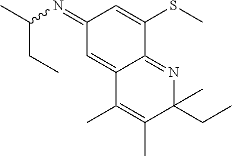 [SMILES: CCC(C)N=C1C=C(SC)C2=NC(C)(CC)C(C)=C(C)C2=C1] |
| 30 | 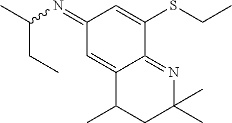 [SMILES: CCSC1=CC(=NC(C)CC)C=C2C1=NC(C)(C)CC2C] | 99 | 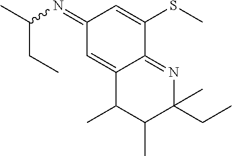 [SMILES: CCC(C)N=C1C=C(SC)C2=NC(C)(CC)C(C)C(C)C2=C1] |
| 31 | 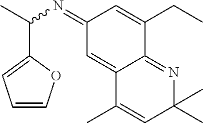 [SMILES: CCC1=CC(=NC(C)c2ccco2)C=C2C(C)=CC(C)(C)N=C12] | 100 | 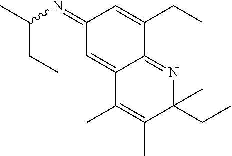 [SMILES: CCC1=CC(=NC(C)CC)C=C2C1=NC(C)(CC)C(C)=C2C] |
| 32 | 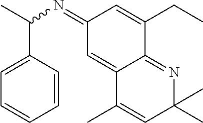 [SMILES: CCC1=CC(=NC(C)c2ccccc2)C=C2C(C)=CC(C)(C)N=C12] | 101 | 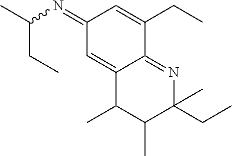 [SMILES: CCC1=CC(=NC(C)CC)C=C2C1=NC(C)(CC)C(C)C2C] |
| 33 | 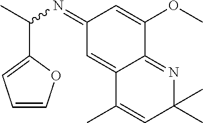 [SMILES: COC1=CC(=NC(C)c2ccco2)C=C2C(C)=CC(C)(C)N=C12] | 102 | 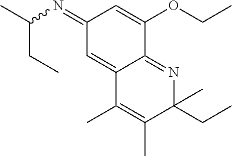 [SMILES: CCOC1=CC(=NC(C)CC)C=C2C1=NC(C)(CC)C(C)=C2C] |
| 34 | 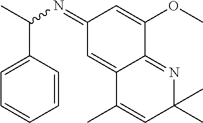 [SMILES: COC1=CC(=NC(C)c2ccccc2)C=C2C(C)=CC(C)(C)N=C12] | 103 | 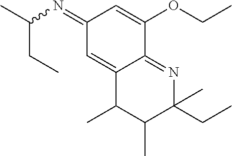 [SMILES: CCOC1=CC(=NC(C)CC)C=C2C1=NC(C)(CC)C(C)C2C] |
| 35 | 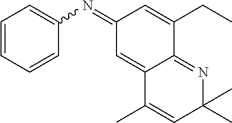 [SMILES: CCC1=CC(=Nc2ccccc2)C=C2C(C)=CC(C)(C)N=C12] | 104 | 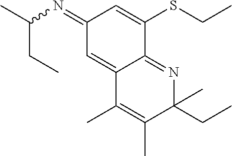 [SMILES: CCSC1=CC(=NC(C)CC)C=C2C1=NC(C)(CC)C(C)=C2C] |
| 36 | 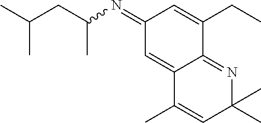 [SMILES: CCC1=CC(=NC(C)CC(C)C)C=C2C(C)=CC(C)(C)N=C12] | 105 | 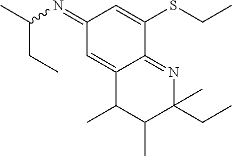 [SMILES: CCSC1=CC(=NC(C)CC)C=C2C1=NC(C)(CC)C(C)C2C] |
| 37 | 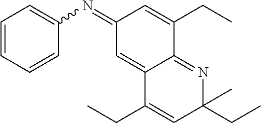 [SMILES: CCC1=CC(C)(CC)N=C2C(CC)=CC(=Nc3ccccc3)C=C12] | 106 | 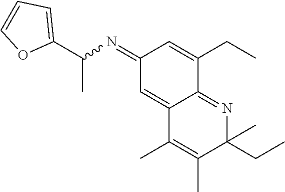 [SMILES: CCC1=CC(=NC(C)c2ccco2)C=C2C1=NC(C)(CC)C(C)=C2C] |
| 38 | 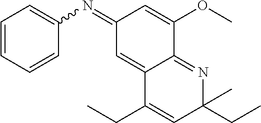 [SMILES: CCC1=CC(C)(CC)N=C2C(OC)=CC(=Nc3ccccc3)C=C12] | 107 | 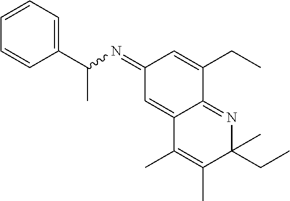 [SMILES: CCC1=CC(=NC(C)c2ccccc2)C=C2C1=NC(C)(CC)C(C)=C2C] |
| 39 | 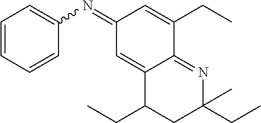 [SMILES: CCC1=CC(=Nc2ccccc2)C=C2C1=NC(C)(CC)CC2CC] | 108 | 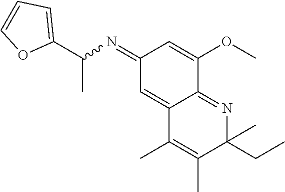 [SMILES: CCC1(C)N=C2C(OC)=CC(=NC(C)c3ccco3)C=C2C(C)=C1C] |
| 40 | 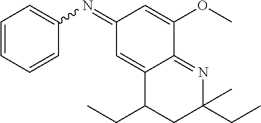 [SMILES: CCC1CC(C)(CC)N=C2C(OC)=CC(=Nc3ccccc3)C=C21] | 109 | 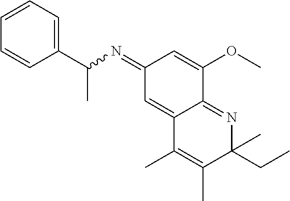 [SMILES: CCC1(C)N=C2C(OC)=CC(=NC(C)c3ccccc3)C=C2C(C)=C1C] |
| 41 | 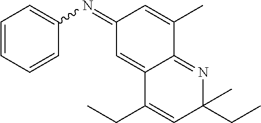 [SMILES: CCC1=CC(C)(CC)N=C2C(C)=CC(=Nc3ccccc3)C=C12] | 110 | 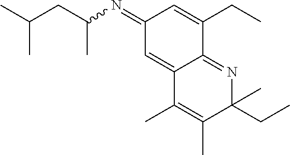 [SMILES: CCC1=CC(=NC(C)CC(C)C)C=C2C1=NC(C)(CC)C(C)=C2C] |
| 42 | 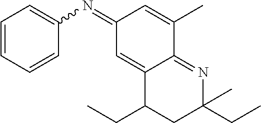 [SMILES: CCC1CC(C)(CC)N=C2C(C)=CC(=Nc3ccccc3)C=C21] | 111 | 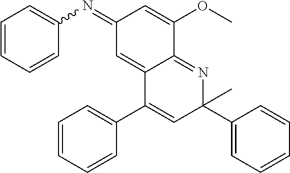 [SMILES: COC1=CC(=Nc2ccccc2)C=C2C(c3ccccc3)=CC(C)(c3ccccc3)N=C12] |
| 43 | 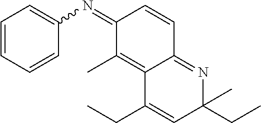 [SMILES: CCC1=CC(C)(CC)N=C2C=CC(=Nc3ccccc3)C(C)=C12] | 112 | 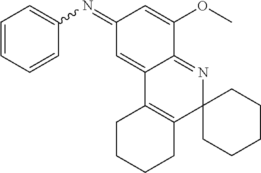 [SMILES: COC1=CC(=Nc2ccccc2)C=C2C1=NC1(CCCCC1)C1=C2CCCC1] |
| 44 | 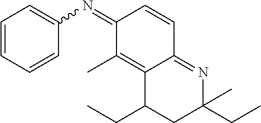 [SMILES: CCC1CC(C)(CC)N=C2C=CC(=Nc3ccccc3)C(C)=C21] | 113 | 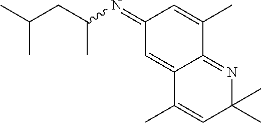 [SMILES: CC1=CC(C)(C)N=C2C(C)=CC(=NC(C)CC(C)C)C=C12] |
| 45 | 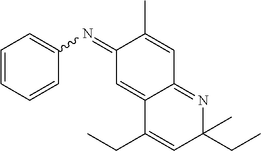 [SMILES: CCC1=CC(C)(CC)N=C2C=C(C)C(=Nc3ccccc3)C=C12] | 114 | 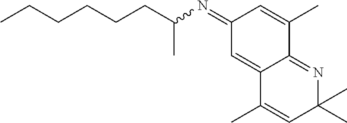 [SMILES: CCCCCCC(C)N=C1C=C(C)C2=NC(C)(C)C=C(C)C2=C1] |
| 46 | 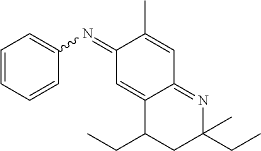 [SMILES: CCC1CC(C)(CC)N=C2C=C(C)C(=Nc3ccccc3)C=C21] | 115 | 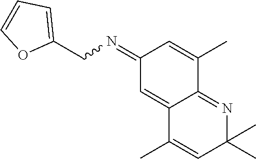 [SMILES: CC1=CC(C)(C)N=C2C(C)=CC(=NCc3ccco3)C=C12] |
| 47 | 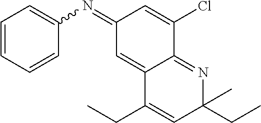 [SMILES: CCC1=CC(C)(CC)N=C2C(Cl)=CC(=Nc3ccccc3)C=C12] | 116 | 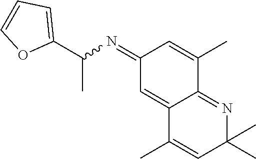 [SMILES: CC1=CC(C)(C)N=C2C(C)=CC(=NC(C)c3ccco3)C=C12] |
| 48 | 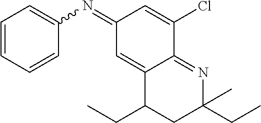 [SMILES: CCC1CC(C)(CC)N=C2C(Cl)=CC(=Nc3ccccc3)C=C21] | 117 | 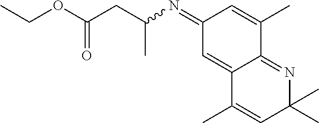 [SMILES: CCOC(=O)CC(C)N=C1C=C(C)C2=NC(C)(C)C=C(C)C2=C1] |
| 49 | 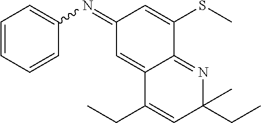 [SMILES: CCC1=CC(C)(CC)N=C2C(SC)=CC(=Nc3ccccc3)C=C12] | 118 | 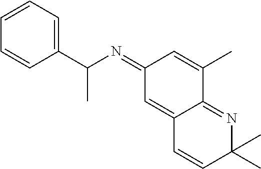 [SMILES: CC1=C/C(=N/C(C)c2ccccc2)C=C2C=CC(C)(C)N=C12] |
| 50 | 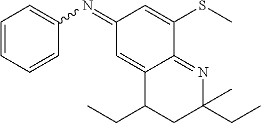 [SMILES: CCC1CC(C)(CC)N=C2C(SC)=CC(=Nc3ccccc3)C=C21] | 119 | 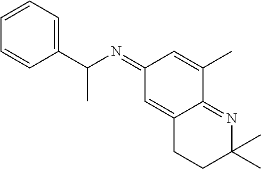 [SMILES: CC1=C/C(=N/C(C)c2ccccc2)C=C2CCC(C)(C)N=C12] |
| 51 | 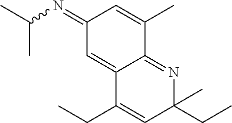 [SMILES: CCC1=CC(C)(CC)N=C2C(C)=CC(=NC(C)C)C=C12] | 120 | 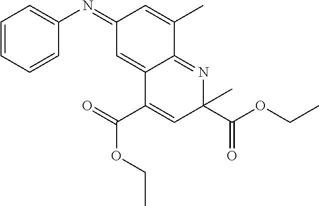 [SMILES: CCOC(=O)C1=CC(C)(C(=O)OCC)N=C2C(C)=C/C(=N/c3ccccc3)C=C12] |
| 52 | 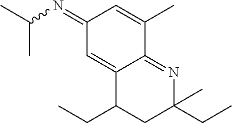 [SMILES: CCC1CC(C)(CC)N=C2C(C)=CC(=NC(C)C)C=C21] | 121 | 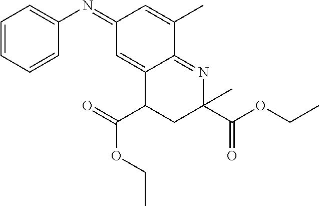 [SMILES: CCOC(=O)C1CC(C)(C(=O)OCC)N=C2C(C)=C/C(=N/c3ccccc3)C=C21] |
| 53 | 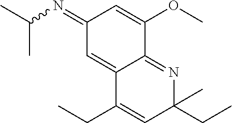 [SMILES: CCC1=CC(C)(CC)N=C2C(OC)=CC(=NC(C)C)C=C12] | 122 | 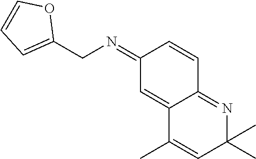 [SMILES: CC1=CC(C)(C)N=C2C=C/C(=N/Cc3ccco3)C=C12] |
| 54 | 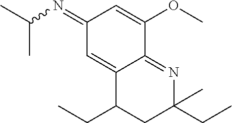 [SMILES: CCC1CC(C)(CC)N=C2C(OC)=CC(=NC(C)C)C=C21] | 123 | 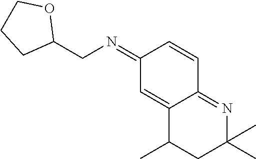 [SMILES: CC1CC(C)(C)N=C2C=C/C(=N/CC3CCCO3)C=C21] |
| 55 | 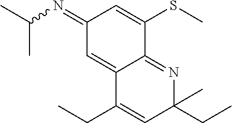 [SMILES: CCC1=CC(C)(CC)N=C2C(SC)=CC(=NC(C)C)C=C12] | 124 | 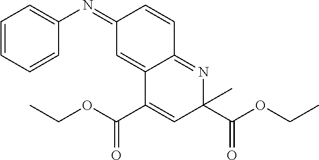 [SMILES: CCOC(=O)C1=CC(C)(C(=O)OCC)N=C2C=C/C(=N/c3ccccc3)C=C12] |
| 56 | 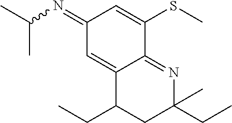 [SMILES: CCC1CC(C)(CC)N=C2C(SC)=CC(=NC(C)C)C=C21] | 125 | 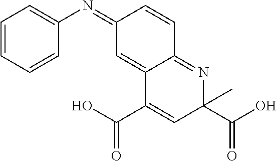 [SMILES: CC1(C(=O)O)C=C(C(=O)O)C2=C/C(=N\c3ccccc3)C=CC2=N1] |
| 57 | 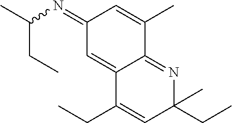 [SMILES: CCC1=CC(C)(CC)N=C2C(C)=CC(=NC(C)CC)C=C12] | 126 | 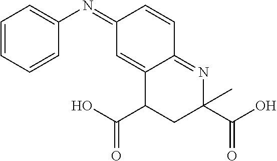 [SMILES: CC1(C(=O)O)CC(C(=O)O)C2=C/C(=N\c3ccccc3)C=CC2=N1] |
| 58 | 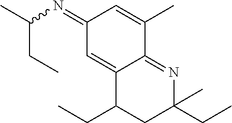 [SMILES: CCC(C)N=C1C=C(C)C2=NC(C)(CC)CC(CC)C2=C1] | 127 | 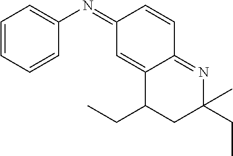 [SMILES: CCC1CC(C)(CC)N=C2C=C/C(=N/c3ccccc3)C=C21] |
| 59 | 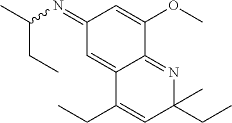 [SMILES: CCC1=CC(C)(CC)N=C2C(OC)=CC(=NC(C)CC)C=C12] | 128 | 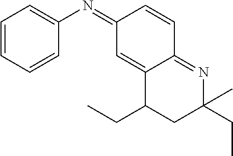 [SMILES: CCC1CC(C)(CC)N=C2C=C/C(=N/c3ccccc3)C=C21] |
| 60 | 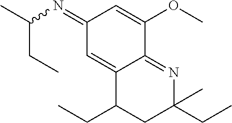 [SMILES: CCC(C)N=C1C=C(OC)C2=NC(C)(CC)CC(CC)C2=C1] | 129 | 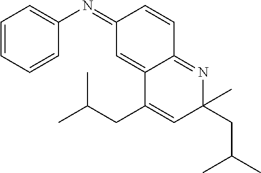 [SMILES: CC(C)CC1=CC(C)(CC(C)C)N=C2C=C/C(=N/c3ccccc3)C=C12] |
| 61 | 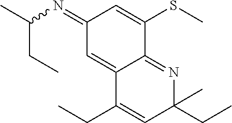 [SMILES: CCC1=CC(C)(CC)N=C2C(SC)=CC(=NC(C)CC)C=C12] | 130 | 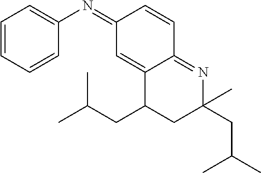 [SMILES: CC(C)CC1CC(C)(CC(C)C)N=C2C=C/C(=N/c3ccccc3)C=C21] |
| 62 | 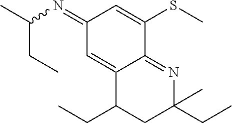 [SMILES: CCC(C)N=C1C=C(SC)C2=NC(C)(CC)CC(CC)C2=C1] | 131 | 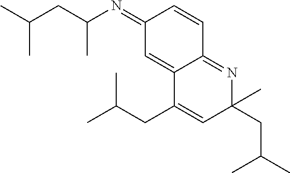 [SMILES: CC(C)CC1=CC(C)(CC(C)C)N=C2C=C/C(=N/C(C)CC(C)C)C=C12] |
| 63 | 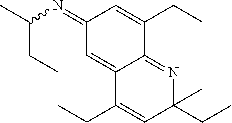 [SMILES: CCC1=CC(C)(CC)N=C2C(CC)=CC(=NC(C)CC)C=C12] | 132 | 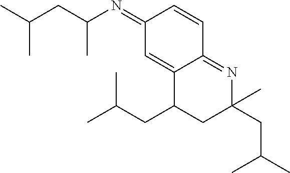 [SMILES: CC(C)CC(C)/N=C1/C=CC2=NC(C)(CC(C)C)CC(CC(C)C)C2=C1] |
| 64 | 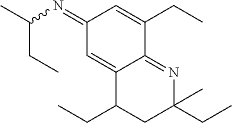 [SMILES: CCC1=CC(=NC(C)CC)C=C2C1=NC(C)(CC)CC2CC] | 133 | 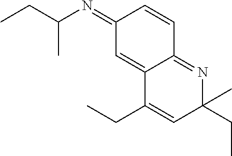 [SMILES: CCC1=CC(C)(CC)N=C2C=C/C(=N/C(C)CC)C=C12] |
| 65 | 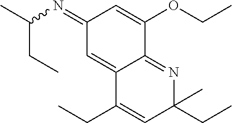 [SMILES: CCOC1=CC(=NC(C)CC)C=C2C(CC)=CC(C)(CC)N=C12] | 134 | 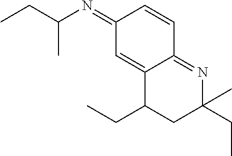 [SMILES: CCC(C)/N=C1/C=CC2=NC(C)(CC)CC(CC)C2=C1] |
| 66 | 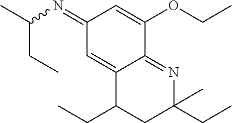 [SMILES: CCOC1=CC(=NC(C)CC)C=C2C1=NC(C)(CC)CC2CC] | 135 | 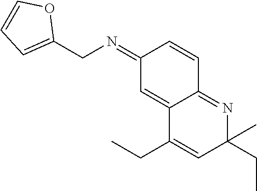 [SMILES: CCC1=CC(C)(CC)N=C2C=C/C(=N/Cc3ccco3)C=C12] |
| 67 | 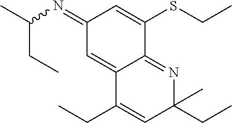 [SMILES: CCSC1=CC(=NC(C)CC)C=C2C(CC)=CC(C)(CC)N=C12] | 136 | 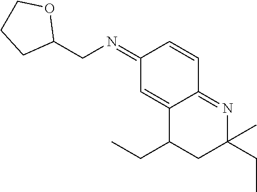 [SMILES: CCC1CC(C)(CC)N=C2C=C/C(=N/CC3CCCO3)C=C21] |
| 68 | 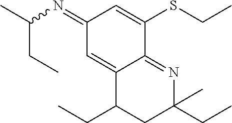 [SMILES: CCSC1=CC(=NC(C)CC)C=C2C1=NC(C)(CC)CC2CC] | 137 | 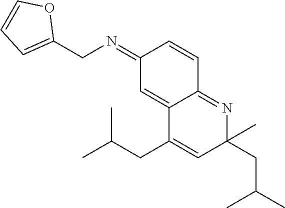 [SMILES: CC(C)CC1=CC(C)(CC(C)C)N=C2C=C/C(=N/Cc3ccco3)C=C12] |
| 69 | 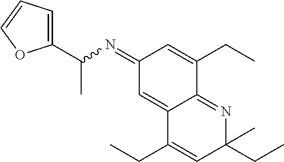 [SMILES: CCC1=CC(C)(CC)N=C2C(CC)=CC(=NC(C)c3ccco3)C=C12] | 138 | 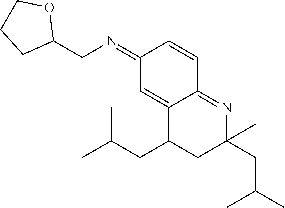 [SMILES: CC(C)CC1CC(C)(CC(C)C)N=C2C=C/C(=N/CC3CCCO3)C=C21] |

In another embodiment, Compounds of the Disclosure are compounds having Formula (VIII):

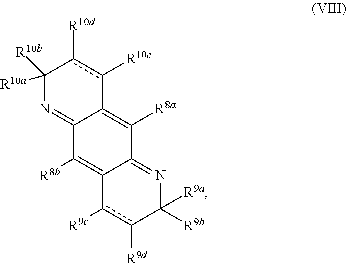

or a salt, solvate, or stereoisomer thereof, wherein:
   each [symbol] is independently a single or a double bond;
   R^{8a} and R^{8b} are independently selected from the group consisting of hydrogen, halogen, C₁-C₉ alkyl, C₁-C₉ haloalkyl, C₃-C₈ cycloalkyl, C₁-C₈ alkoxy, C₁-C₈ alkylthio, —C(═O)OR⁴, —OC(═O)R⁴, —C(═O)NR⁴R⁵, —NR⁴C(═O)R⁵, —NR⁴R⁵, —OH, —SH, —SC(═O)R⁴, —SC(═O)SR⁴, —SC(═O)NR⁴R⁵, —NR⁴C(═O)SR⁵, —NR⁴C(═S)SR⁵, 4- to 6-membered heterocyclyl, optionally substituted phenyl, optionally substituted 5- or 6-membered heteroaryl, aryloxy, arylthio, and arylamino;
   R^{9a} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and

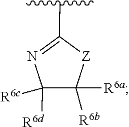

and
R^{6b} is selected from the group consisting of hydrogen, halogen, C₁-C₉ alkyl, and optionally substituted phenyl; or
R^{9a} and R^{6b} taken together with the carbon atom to which they are attached form a C₃-C₁₂ cycloalkyl;
R^{6c} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and

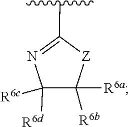

and
R^{6d} is selected from the group consisting of hydrogen, halogen, C₁-C₉ alkyl, and optionally substituted phenyl; or
R^{6c} and R^{6d} taken together with the two carbon atoms to which they are attached form a C₃-C₁₂ cycloalkyl;
R^{10a} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and

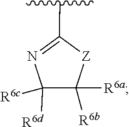

and
R^{10b} is selected from the group consisting of hydrogen, halogen, C₁-C₉ alkyl, and optionally substituted phenyl; or
R^{10a} and R^{10b} taken together with the carbon atom to which they are attached form a C₃-C₁₂ cycloalkyl;
R^{10c} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and

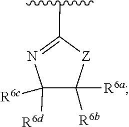

and
R^{10d} is selected from the group consisting of hydrogen, halogen, C₁-C₉ alkyl, and optionally substituted phenyl; or
R^{10c} and R^{10d} taken together with the two carbon atoms to which they are attached form a C₃-C₁₂ cycloalkyl;
Z at each occurrence is independently selected from the group consisting of —O—, —S—, and —NR⁷—;
R⁴ at each occurrence is independently selected from the group consisting of hydrogen, C₁-C₉ alkyl, C₃-C₆ cycloalkyl, and optionally substituted phenyl; and
R⁵ at each occurrence is independently selected from the group consisting of hydrogen, C₁-C₉ alkyl, C₃-C₆ cycloalkyl, and optionally substituted phenyl;
R^{6a}, R^{6b}, R^{6c}, and R^{6d} at each occurrence is independently are independently selected from the group consisting of hydrogen, C₁-C₁₂ alkyl, and optionally substituted phenyl; and
R⁷ at each occurrence is independently selected from the group consisting of hydrogen and C₁-C₆ alkyl.

In another embodiment, Compounds of the Disclosure are compounds having Formula (IX):

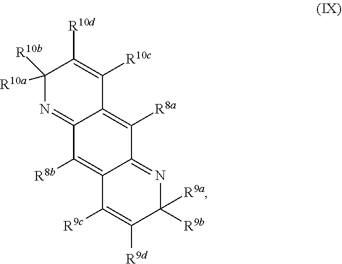

or a salt, solvate, or stereoisomer thereof, wherein R^{8a}, R^{8b}, R^{9a}, R^{9b}, R^{9c}, R^{9d}, R^{10a}, R^{10b}, R^{10c}, and R^{10d} are as defined in connection with Formula (VIII).

In another embodiment, Compounds of the Disclosure are compounds having Formula (X):

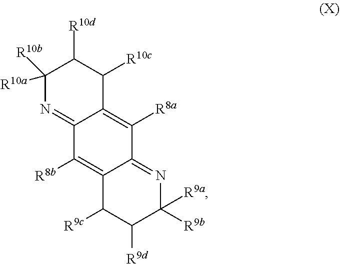

or a salt, solvate, or stereoisomer thereof, wherein R^{8a}, R^{8b}, R^{9a}, R^{9b}, R^{9c}, R^{9d}, R^{10a}, R^{10b}, R^{10c}, and R^{10d} are as defined in connection with Formula (VIII).

In some embodiments, Compounds of the Disclosure are compounds having any one of Formulae (VIII)-(X), wherein R^{8a} and R^{8b} are hydrogen.

In some embodiments, Compounds of the Disclosure are compounds having any one of Formulae (VIII)-(X), wherein R^{8a} and R^{8b} are methyl.

In some embodiments, Compounds of the Disclosure are compounds having any one of Formulae (VIII)-(X), wherein R^{9a}, R^{9b}, and R^{9c} are independently selected from the group consisting of methyl, ethyl, propyl, iso-propyl, n-butyl, sec-butyl, iso-butyl, tert-butyl, pentyl, hexyl, heptyl, octyl, and nonyl.

In some embodiments, Compounds of the Disclosure are compounds having any one of Formulae (VIII)-(X), wherein R^{9a}, R^{9b}, and R^{9c} are methyl, ethyl, or iso-butyl.

In some embodiments, Compounds of the Disclosure are compounds having any one of Formulae (VIII)-(X), wherein R^{9a} and R^{9c} are methyl.

In some embodiments, Compounds of the Disclosure are compounds having any one of Formulae (VIII)-(X), wherein R^{9a} and R^{9c} are ethyl.

In some embodiments, Compounds of the Disclosure are compounds having any one of Formulae (VIII)-(X), wherein R^{9a} and R^{9c} are iso-butyl.

In some embodiments, Compounds of the Disclosure are compounds having any one of Formulae (VIII)-(X), wherein R^{6b} is methyl.

In some embodiments, Compounds of the Disclosure are compounds having any one of Formulae (VIII)-(X), wherein R^{9a} is —C(═O)OR⁴, —C(═O)NR⁴R⁵, —C(═O)SR⁴, —CH₂OR⁴, —CH₂OC(═O)R⁴, or

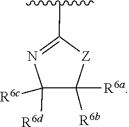

In some embodiments, Compounds of the Disclosure are compounds having any one of Formulae (VIII)-(X), wherein R^{9a} is —C(═O)OR⁴. In some embodiments, R^{9a} is —C(═O)OEt.

In some embodiments, Compounds of the Disclosure are compounds having any one of Formulae (VIII)-(X), wherein R^{9a} is —C(═O)NR⁴R⁵.

In some embodiments, Compounds of the Disclosure are compounds having any one of Formulae (VIII)-(X), wherein R^{9a} is —C(═O)SR⁴.

In some embodiments, Compounds of the Disclosure are compounds having any one of Formulae (VIII)-(X), wherein R^{9a} is —CH₂OR⁴.

In some embodiments, Compounds of the Disclosure are compounds having any one of Formulae (VIII)-(X), wherein R^{9a} is —CH₂OC(═O)R⁴.

In some embodiments, Compounds of the Disclosure are compounds having any one of Formulae (VIII)-(X), wherein R^{9a} is

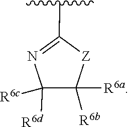

In some embodiments, Compounds of the Disclosure are compounds having any one of Formulae (VIII)-(X), wherein R^{9c} is —C(═O)OR⁴. In some embodiments, R^{9c} is —C(═O)OEt.

In some embodiments, Compounds of the Disclosure are compounds having any one of Formulae (VIII)-(X), wherein R^{9c} is —C(═O)NR⁴R⁵.

In some embodiments, Compounds of the Disclosure are compounds having any one of Formulae (VIII)-(X), wherein R^{9c} is —C(═O)SR⁴.

In some embodiments, Compounds of the Disclosure are compounds having any one of Formulae (VIII)-(X), wherein R^{9c} is —CH₂OR⁴.

In some embodiments, Compounds of the Disclosure are compounds having any one of Formulae (VIII)-(X), wherein R^{9c} is —CH₂OC(═O)R⁴.

In some embodiments, Compounds of the Disclosure are compounds having any one of Formulae (VIII)-(X), wherein R^{9c} is

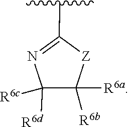

In some embodiments, Compounds of the Disclosure are compounds having any one of Formulae (VIII)-(X), wherein R^{10a}, R^{10b}, and R^{10c} are independently selected from the group consisting of methyl, ethyl, propyl, iso-propyl, n-butyl, sec-butyl, iso-butyl, tert-butyl, pentyl, hexyl, heptyl, octyl, and nonyl.

In some embodiments, Compounds of the Disclosure are compounds having any one of Formulae (VIII)-(X), wherein R^{10a}, R^{10b}, and R^{10c} are methyl, ethyl, or iso-butyl.

In some embodiments, Compounds of the Disclosure are compounds having any one of Formulae (VIII)-(X), wherein R^{10a} and R^{10c} are methyl.

In some embodiments, Compounds of the Disclosure are compounds having any one of Formulae (VIII)-(X), wherein R^{10a} and R^{10c} are ethyl.

In some embodiments, Compounds of the Disclosure are compounds having any one of Formulae (VIII)-(X), wherein R^{10a} and R^{10c} are iso-butyl.

In some embodiments, Compounds of the Disclosure are compounds having any one of Formulae (VIII)-(X), wherein R^{10b} is methyl.

In some embodiments, Compounds of the Disclosure are compounds having any one of Formulae (VIII)-(X), wherein R^{10a} is —C(═O)OR⁴, —C(═O)NR⁴R⁵, —C(═O)SR⁴, —CH₂OR⁴, —CH₂OC(═O)R⁴, or

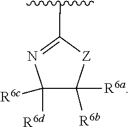

In some embodiments, Compounds of the Disclosure are compounds having any one of Formulae (VIII)-(X), wherein R^{10a} is —C(═O)OR⁴. In some embodiments, R^{10a} is —C(═O)OEt.

In some embodiments, Compounds of the Disclosure are compounds having any one of Formulae (VIII)-(X), wherein R^{10a} is —C(═O)NR⁴R⁵.

In some embodiments, Compounds of the Disclosure are compounds having any one of Formulae (VIII)-(X), wherein R^{10a} is —C(═O)SR⁴.

In some embodiments, Compounds of the Disclosure are compounds having any one of Formulae (VIII)-(X), wherein R^{10a} is —CH₂OR⁴.

In some embodiments, Compounds of the Disclosure are compounds having any one of Formulae (VIII)-(X), wherein R^{10a} is —CH₂OC(═O)R⁴.

In some embodiments, Compounds of the Disclosure are compounds having any one of Formulae (VIII)-(X), wherein R^{10a} is

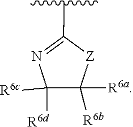

In some embodiments, Compounds of the Disclosure are compounds having any one of Formulae (VIII)-(X), wherein R^{10c} is —C(═O)OR⁴. In some embodiments, R^{10c} is —C(═O)OEt.

In some embodiments, Compounds of the Disclosure are compounds having any one of Formulae (VIII)-(X), wherein R^{10c} is —C(═O)NR⁴R⁵.

In some embodiments, Compounds of the Disclosure are compounds having any one of Formulae (VIII)-(X), wherein R^{10c} is —C(═O)SR⁴.

In some embodiments, Compounds of the Disclosure are compounds having any one of Formulae (VIII)-(X), wherein R^{10c} is —CH₂OR⁴.

In some embodiments, Compounds of the Disclosure are compounds having any one of Formulae (VIII)-(X), wherein R^{10c} is —CH₂OC(═O)R⁴.

In some embodiments, Compounds of the Disclosure are compounds having any one of Formulae (VIII)-(X), wherein R^{10c} is

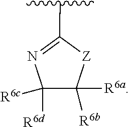

In some embodiments, Compounds of the Disclosure are compounds having any one of Formulae (VIII)-(X), wherein Z is —O—. In some embodiments, Z is —S—. In some embodiments, Z is —NR⁷—. In some embodiments, R⁷ is hydrogen. In some embodiments, R^{6a}, R^{6b}, R^{6c}, and R^{6d} are hydrogen.

In some embodiments, Compounds of the Disclosure are compounds having any one of Formulae (VIII)-(X), wherein each R⁴ is hydrogen or C₁-C₉ alkyl.

In some embodiments, Compounds of the Disclosure are compounds having any one of Formulae (VIII)-(X), wherein each R⁴ is ethyl.

In some embodiments, Compounds of the Disclosure are compounds having any one of Formulae (VIII)-(X), wherein each R⁴ is hydrogen.

In some embodiments, Compounds of the Disclosure are compounds having any one of Formulae (VIII)-(X), wherein R^{9a} and R^{10a} are the same, e.g., R^{9a} and R^{10a} are both methyl, both ethyl, or both iso-butyl.

In some embodiments, Compounds of the Disclosure are any one or more of the compounds of Table 1B, or a salt or solvate thereof.

TABLE 1B

| Compound No. | Structure |
|---|---|
| 139 | 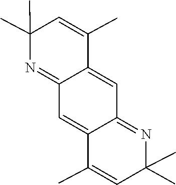 |
| 140 | 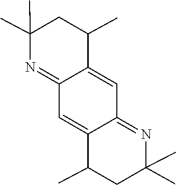 |
| 141 | 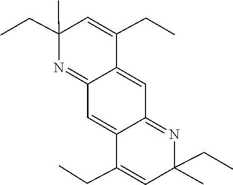 [SMILES: CCC1=CC(C)(CC)N=c2cc3c(cc21)=NC(C)(CC)C=C3CC] |
| 142 | 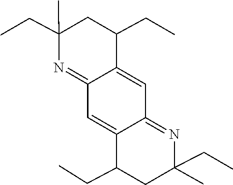 [SMILES: CCC1CC(C)(CC)N=c2cc3c(cc21)=NC(C)(CC)CC3CC] |
| 143 | 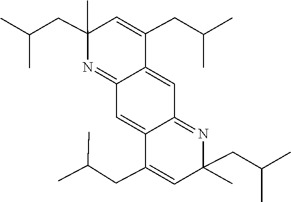 [SMILES: CC(C)CC1=CC(C)(CC(C)C)N=c2cc3c(cc21)=NC(C)(CC(C)C)C=C3CC(C)C] |
| 144 | 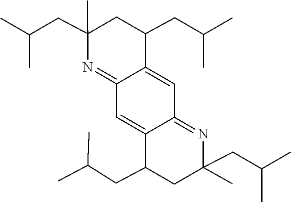 [SMILES: CC(C)CC1CC(C)(CC(C)C)N=c2cc3c(cc21)=NC(C)(CC(C)C)CC3CC(C)C] |
| 145 | 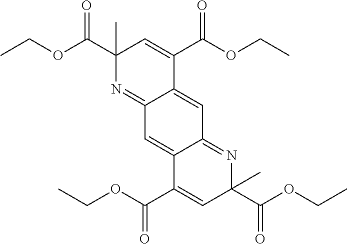 [SMILES: CCOC(=O)C1=CC(C)(C(=O)OCC)N=c2cc3c(cc21)=NC(C)(C(=O)OCC)C=C3C(=O)OCC] |
| 146 | 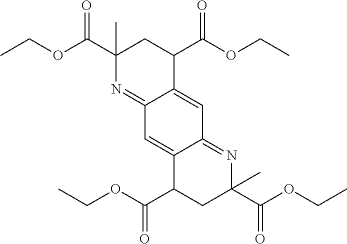 [SMILES: CCOC(=O)C1CC(C)(C(=O)OCC)N=c2cc3c(cc21)=NC(C)(C(=O)OCC)CC3C(=O)OCC] |
| 147 | 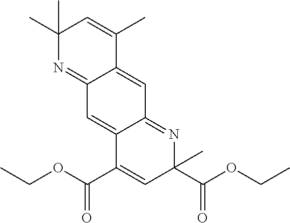 [SMILES: CCOC(=O)C1=CC(C)(C(=O)OCC)N=c2cc3c(cc21)=NC(C)(C)C=C3C] |
| 148 | 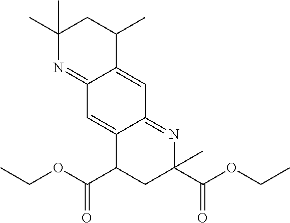 [SMILES: CCOC(=O)C1CC(C)(C(=O)OCC)N=c2cc3c(cc21)=NC(C)(C)CC3C] |
| 149 | 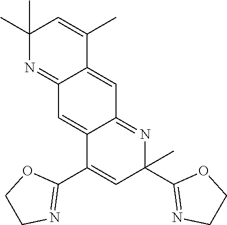 [SMILES: CC1=CC(C)(C)N=c2cc3c(cc21)=NC(C)(C1=NCCO1)C=C3C1=NCCO1] |
| 150 | 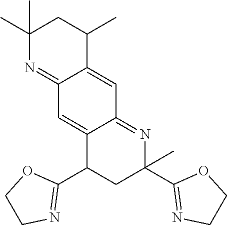 [SMILES: CC1CC(C)(C)N=c2cc3c(cc21)=NC(C)(C1=NCCO1)CC3C1=NCCO1] |
| 151 | 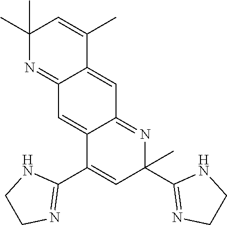 [SMILES: CC1=CC(C)(C)N=c2cc3c(cc21)=NC(C)(C1=NCCN1)C=C3C1=NCCN1] |
| 152 | 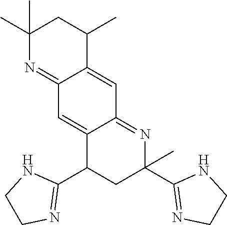 [SMILES: CC1CC(C)(C)N=c2cc3c(cc21)=NC(C)(C1=NCCN1)CC3C1=NCCN1] |
| 153 | 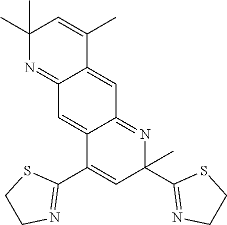 [SMILES: CC1=CC(C)(C)N=c2cc3c(cc21)=NC(C)(C1=NCCS1)C=C3C1=NCCS1] |
| 154 | 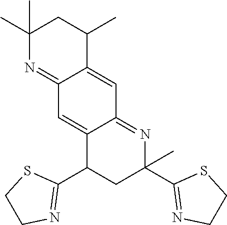 [SMILES: CC1CC(C)(C)N=c2cc3c(cc21)=NC(C)(C1=NCCS1)CC3C1=NCCS1] |

Compounds of the Disclosure can be useful as antidegradants, e.g., antiozonants and/or antioxidants, as additives in lubricants, and as additives in combustible fuels.

Compounds of the Disclosure can be prepared by dehydrogenation of the corresponding quinolineamine or bis(quinolineamine) as shown in Schemes 1 and 2. The dehydrogenation can be carried out using any method known in the art, i.e. treatment with an oxidant such as sodium hypochlorite, O₂, H₂O₂ or the like in a solvent such as hexane, heptane, acetonitrile or the like, and optionally using a catalyst such as tetrabutylammonium bromide or the like.

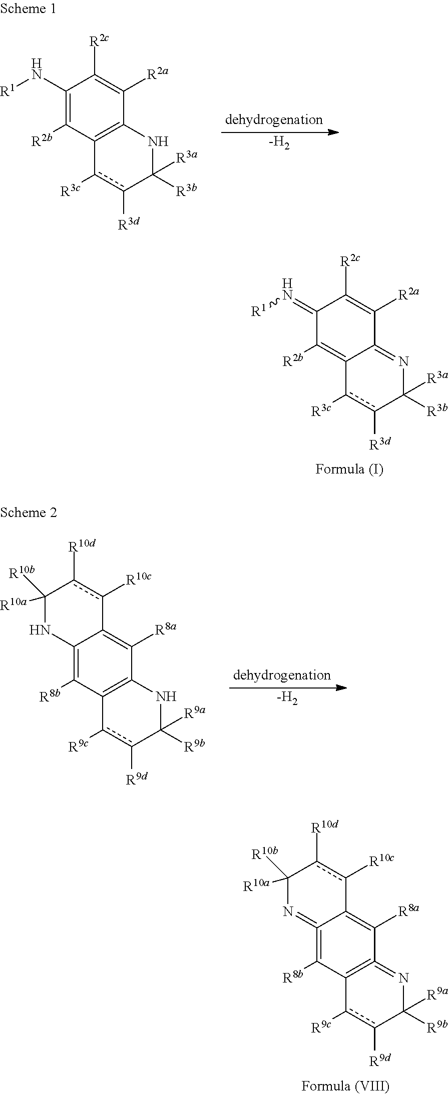

### Definitions

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., C₁-C₃ alkyl such as methyl, ethyl, propyl, or isopropyl; a C₁-C₄ alkyl such as methyl, ethyl, propyl, isopropyl, n-butyl, isobutyl, sec-butyl, or t-butyl; and so on. In one embodiment the alkyl is a straight-chain alkyl. In another embodiment, the alkyl is a branched-chain alkyl. In one embodiment, the alkyl is a C₁-C₈ alkyl. In another embodiment, the alkyl is a C₁-C₆ alkyl. In another embodiment, the alkyl is a C₁-C₄ alkyl. In another embodiment, the alkyl is a C₁-C₃ alkyl. Nonlimiting exemplary C₁-C₁₂ alkyl groups include methyl, ethyl, propyl, isopropyl, butyl, secbutyl, tert-butyl, iso-butyl, 3pentyl, hexyl, heptyl, octyl, nonyl, and decyl.

The term “optionally substituted alkyl” as used herein by itself or as part of another group refers to an alkyl that is either unsubstituted or substituted with one, two, or three substituents, wherein the substituents are each independently nitro, cyano, hydroxyl, amino, (e.g., —NH₂, alkylamino, or dialkylamino), alkoxy, alkylthio, alkylcarbonyl, cycloalkyl, or —C(═O)OR^{1h}, wherein R^{1h} is C₁-C₆ alkyl

The term “halo” or “halogen” as used herein by itself or as part of another group refers to Cl, F, —Br, or —I.

The term “nitro” as used herein by itself or as part of another group refers to NO₂.

The term “cyano” as used herein by itself or as part of another group refers to CN.

The term “hydroxy” as herein used by itself or as part of another group refers to OH.

The term “amino” as used by itself or as part of another group refers to a radical of the formula-NR^{1f}R^{1g}, wherein R^{1f} and R^{1g} are independently hydrogen or alkyl.

In one embodiment, the amino is —NH₂.

In another embodiment, the amino is an “alkylamino,” i.e., an amino group wherein R^{1f} is C₁-C₆ alkyl and R^{1g} is hydrogen. In one embodiment, R^{1f} is C₁-C₄ alkyl. Non-limiting exemplary alkylamino groups include N(H)CH₃ and N(H)CH₂CH₃.

In another embodiment, the amino is a “dialkylamino,” i.e., an amino group wherein R^{1f} and Rig are each independently C₁₋₆ alkyl. In one embodiment, R^{1f} and R^{1g} are each independently C₁-C₄ alkyl. Non-limiting exemplary dialkylamino groups include-N(CH₃) 2 and —N(CH₃) CH₂CH(CH₃) 2.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A nonlimiting exemplary alkylcarbonyl group is —COCH₃.

The term “haloalkyl” as used herein by itself or as part of another group refers to an alkyl substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl and the resulting haloalkyl is referred to as a “C₁-C₆ haloalkyl.” In another embodiment, the alkyl is a C₁-C₄ alkyl and the resulting haloalkyl is referred to as a “C₁-C₄ haloalkyl.” In another embodiment, the alkyl group is a C₁ or C₂ alkyl. Non-limiting exemplary haloalkyl groups include fluoromethyl, difluoromethyl, trifluoromethyl, pentafluoroethyl, 1,1difluoroethyl, 2,2-difluoroethyl, 2,2,2trifluoroethyl, 3,3,3-trifluoropropyl, 4,4,4trifluorobutyl, and trichloromethyl groups.

The term “alkoxy” as used herein by itself or as part of another group refers to an alkyl attached to a terminal oxygen atom. In one embodiment, the alkyl is a C₁-C₆ alkyl, and the resulting alkoxy is referred to as a “C₁-C₆ alkoxy.” In another embodiment, the alkyl is a C₁-C₄ alkyl group and thus the resulting alkoxy is referred to as a “C₁-C₄ alkoxy.” Non-limiting exemplary alkoxy groups include methoxy, ethoxy, and tert-butoxy.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one, two, three, four, or five substituents, wherein the substituents are each independently halo, nitro, cyano, hydroxyl, amino, (e.g., —NH₂, alkylamino, or dialkylamino), alkoxy, alkylthio, alkylcarbonyl, alkyl, or cycloalkyl, and the like.

The term “aryloxy” as used herein by itself or as part of another group refers to an optionally substituted aryl attached to a terminal oxygen atom. In one embodiment, the optionally substituted aryl is an optionally substituted phenyl. Non-limiting exemplary aryloxy groups include-O-phenyl.

The term “arylthio” as used herein by itself or as part of another group refers to an optionally substituted aryl attached to a terminal sulfur atom. In one embodiment, the optionally substituted aryl is an optionally substituted phenyl. Non-limiting exemplary aryloxy groups include-S-phenyl.

The term “arylamino” as used herein by itself or as part of another group refers to an optionally substituted aryl attached to a terminal —NH— group. In one embodiment, the optionally substituted aryl is an optionally substituted phenyl. Non-limiting exemplary aryloxy groups include-NH-phenyl.

The term “alkylthio” as used herein by itself or as part of another group refers to an alkyl group attached to a terminal sulfur atom. In one embodiment, the alkyl is a C₁-C₆ alkyl, and the resulting alkylthio is referred to as a “C₁-C₆ alkylthio.” In one embodiment, the alkyl group is a C₁-C₄ alkyl group and the resulting alkylthio is referred to as a “C₁-C₄ alkylthio.” Non-limiting exemplary alkylthio groups include-SCH₃, and —SCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃C₁₂ cycloalkyl, or the number of carbons designated, e.g., a C₃-C₆ cycloalkyl such a cyclopropyl, cyclobutyl, cyclopentyl, and cyclohexyl. In one embodiment, the cycloalkyl is bicyclic, i.e., it has two rings. In another embodiment, the cycloalkyl is monocyclic, i.e., it has one ring. In another embodiment, the cycloalkyl is a C₃C₈ cycloalkyl. In another embodiment, the cycloalkyl is a C₃-C₆ cycloalkyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃C₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted phenyl” as used herein by itself or as part of another group refers to phenyl that is either unsubstituted or substituted with one to five substituents, wherein the substituents are, e.g., each independently halo, nitro, cyano, hydroxyl, amino, (e.g., —NH₂, alkylamino, or dialkylamino), alkoxy, alkylthio, alkylcarbonyl, alkyl, or cycloalkyl.

The term “heterocyclo” as used herein by itself or as part of another group refers to saturated and partially unsaturated, e.g., containing one or two double bonds, monocyclic, bicyclic, or tricyclic groups containing three to eighteen ring members, i.e., a 3- to 18membered heterocyclo, comprising one, two, three, or four heteroatoms. Each heteroatom is independently oxygen, sulfur, or nitrogen. Each sulfur atom may be independently oxidized to give a sulfoxide, i.e., S(═O), or sulfone, i.e., S(═O) 2. The term heterocyclo includes groups wherein one or more CH₂-groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclo also includes groups having fused optionally substituted aryl or optionally substituted heteroaryl groups such as indoline, indolin-2-one, 2,3dihydro-1H-pyrrolo[2,3-c]pyridine, 2,3,4,5-tetrahydro-1H-benzo[d]azepine, or 1,3,4,5-tetrahydro-2H-benzo[d]azepin-2-one. In some embodiments, heterocyclo is a 6-membered ring comprising one nitrogen atom. The heterocyclo may be fused to the rest of the molecule to form a bicyclic group, e.g., 1,2-dihydroquinoline or 1,2,3,4-tetrahydroquinoline.

The term “optionally substituted heterocyclo” as used herein by itself or part of another group refers to a heterocyclo group that is either unsubstituted or substituted with one to four substituents, wherein the substituents are each independently halo, nitro, cyano, hydroxyl, amino, (e.g., —NH₂, alkylamino, or dialkylamino), alkoxy, alkylthio, alkylcarbonyl, alkyl, or cycloalkyl.

The term “heteroaryl” as used herein by itself or as part of another group refers to monocyclic aromatic ring systems having five to six ring members, i.e., a 5 to 6membered heteroaryl, comprising one, two, three, four, or five heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl has 5 ring atoms, e.g., furyl, a 5-membered heteroaryl having four carbon atoms and one oxygen atom. In another embodiment, the heteroaryl has 6 ring atoms, e.g., pyridyl, a 6membered heteroaryl having five carbon atoms and one nitrogen atom. Non-limiting exemplary heteroaryl groups include thienyl, furyl, pyranyl, 2H-pyrrolyl, pyrrolyl, imidazolyl, pyrazolyl, pyridyl, pyrazinyl, pyrimidinyl, pyridazinyl, thiazolyl, isothiazolyl, and isoxazolyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes Noxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxyl, amino, (e.g., —NH₂, alkylamino, or dialkylamino), alkoxy, alkylthio, alkylcarbonyl, alkyl, or cycloalkyl.

As used herein, the term “stereoisomers” is a general term for all isomers of an individual molecule that differ only in the orientation of their atoms in space. It includes enantiomers and isomers of compounds with more than one chiral center that are not mirror images of one another (diastereomers).

In some embodiments, a Compound of the Disclosure comprises a mixture of E and Z stereoisomers as illustrated for Compound No. 1 below:

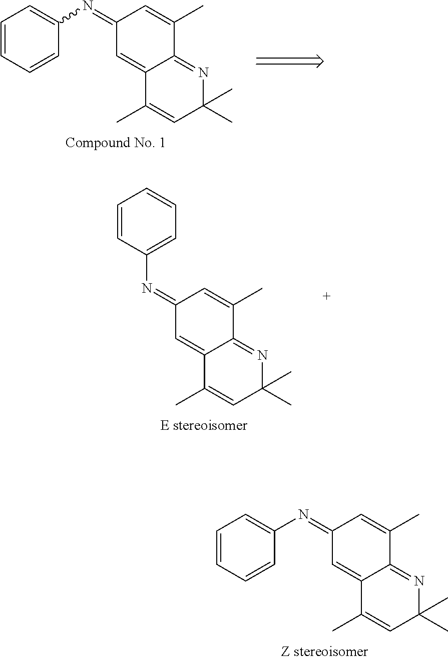 [SMILES: CC1=CC(C)(C)N=C2C(C)=CC(=Nc3ccccc3)C=C12 ; CC1=CC(C)(C)N=C2C(C)=C/C(=N\c3ccccc3)C=C12 ; CC1=CC(C)(C)N=C2C(C)=C/C(=N/c3ccccc3)C=C12]

In some embodiments, a Compound of the Disclosure comprises a mixture of E and Z stereoisomers, wherein the E:Z ratio is 500:1 to 1:500. In some embodiments, the E:Z ratio is 100:1 to 1:100. In some embodiments, the E:Z ratio is 10:1 to 1:10. In some embodiments, the E:Z ratio is 5:1 to 1:5. In some embodiments, the E:Z ratio is 3:1 to 1:3. In some embodiments, the E:Z ratio is 2:1 to 1:2. In some embodiments, the E:Z ratio is about 10:1, about 9:1, about 8:1, about 7:1, about 6:1, about 5:1, about 4:1, about 3:1, or about 2:1. In some embodiments, the E:Z ratio is about 1:10, about 1:9, about 1:8, about 1:7, about 1:6, about 1:5, about 1:4, about 1:3, or about 1:2. In some embodiments, the E:Z ratio is about 1:1.

In some embodiments, a Compound of the Disclosure comprises only the E stereoisomer, i.e., there is no detectable amount of the Z stereoisomer as measured by HPLC.

In some embodiments, a Compound of the Disclosure comprises only the Z stereoisomer, i.e., there is no detectable amount of the E stereoisomer as measured by HPLC.

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R-S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography, or optical polarimetry.

In thin layer chromatography (TLC), the term R_{f} stands for retention factor. R_{f} is defined as the distance travelled by an individual component divided by the total distance travelled by the eluent. Its value is always between zero and one.

Salts and solvates, e.g., hydrates, of the Compounds of the Disclosure can also be used in the methods disclosed herein.

The present disclosure encompasses the preparation and use of salts of Compounds of the Disclosure. Salts of Compounds of the Disclosure can be prepared during the final isolation and purification of the compounds or separately by reacting the compound with an acid having a suitable cation. Salts of Compounds of the Disclosure can be acid addition salts formed with acceptable acids. Examples of acids which can be employed to form salts include inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2naphthalenesulfonate, oxalate, pamoate, pectinate, persulfate, 3phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and ptoluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to Compounds of the Disclosure appearing herein is intended to include Compounds of the Disclosure as well as salts, hydrates, or solvates thereof.

The present disclosure encompasses the preparation and use of solvates of Compounds of the Disclosure. The term “solvate” as used herein is a combination, physical association and/or solvation of a compound of the present disclosure with a solvent molecule such as, e.g., a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a solvent, such as water, methanol, and ethanol, and it is intended that the disclosure includes both solvated and unsolvated forms of Compounds of the Disclosure.

One type of solvate is a hydrate. A “hydrate” relates to a particular subgroup of solvates where the solvent molecule is water. Preparation of solvates is known in the art. See, for example, M. Caira et al, J. Pharmaceut. Sci., 93 (3): 601-611 (2004), which describes the preparation of solvates of fluconazole with ethyl acetate and with water. Similar preparation of solvates, hemisolvates, hydrates, and the like are described by van Tonder et al., AAPS Pharm. Sci. Tech., 5 (1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “wt/wt %” as used herein refers to the mass of one component in a composition or blend, e.g., a composition comprising a Compound of the Disclosure and one or more elastomers; or a Composition of the Disclosure and one or more fillers; or a blend comprising two or more elastomers, etc., divided by the combined mass of all components in the composition or blend, times 100. For example, the wt/wt % of a Compound of the Disclosure in a composition comprising 1 kg of the compound, 1 kg natural rubber, and 2 kg of synthetic rubber, is 25 wt/wt % (1 kg/4 kg=0.25×100=25 wt/wt %). The wt/wt % of a Compound of the Disclosure in a composition comprising 1 kg of the compound and 1 kg of carbon black is 50 wt/wt % (1 kg/2 kg=0.50×100=50 wt/wt %). The wt/wt % of a Compound of the Disclosure in a composition comprising 1 kg of the compound, 1 kg natural rubber, 2 kg of synthetic rubber, and 1 kg of carbon black is 20 wt/wt % (1 kg/5 kg=0.20×100=20 wt/wt %). The wt/wt % of natural rubber in a blend of one or more elastomers comprising 20 kg natural rubber and 30 kg synthetic rubber is 40 wt/wt % (20 kg/50 kg=0.40×100=40 wt/wt %).

As used herein, the term “masterbatch” refers to a composition comprising at least one elastomer and a Compound of the Disclosure. In some embodiments, the masterbatch comprises from about 5 wt/wt % to about 95 wt/wt % of at least one elastomer and from about 5 wt/wt % to about 95 wt/wt % of a Compound of the Disclosure. In some embodiments, the masterbatch comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound of the Disclosure. In some embodiments, the masterbatch comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one elastomer.

As used herein, the term “premix” refers to a composition comprising a Compound of the Disclosure and at least one additional component, e.g., a filler, a rubber chemical, a plasticizer, an additional antidegradant, or a combination thereof. In some embodiments, the premix does not comprise an elastomer. In some embodiments, the premix comprises from about 5 wt/wt % to about 95 wt/wt % of a Compound of the Disclosure and from about 5 wt/wt % to about 95 wt/wt % of the least one additional component, e.g., a filler, a rubber chemical, a plasticizer, an additional antidegradant, or a combination thereof. In some embodiments, the premix comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound of the Disclosure. In some embodiments, the premix comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one additional component, e.g., a filler, a rubber chemical, a plasticizer, an additional antidegradant, or a combination thereof.

The term “one or more” as used herein is intended to mean that at least one component in a relevant category of components, e.g. one or more elastomers, or at least one component in a disclosed list, e.g., one or more fluorine, chlorine, bromine, and/or iodine atoms, is present and, in some embodiments, more than one component is present. In some embodiments, one or more means 1 to 50. In some embodiments, one or more means 1 to 40. In some embodiments, one or more means 1 to 30. In some embodiments, one or more means 1 to 20. In some embodiments, one or more means 1 to 10. In some embodiments, one or more means 1 to 5. In some embodiments, one or more means 1 to 3. In some embodiments, one or more means 1 or 2. In some embodiments, one or more means 1.

### Compositions and Methods of Use

In another embodiment, the disclosure provides compositions comprising:
(i) a Compound of the Disclosure; and
(ii) one or more elastomers; or
(iii) one or more fillers; or
(iv) one or more rubber chemicals; or
(v) one or more plasticizers; or
(vi) one or more additional antidegradants; or
(vii) a combination of one or more elastomers, one or more fillers, one or more rubber chemicals, one or more plasticizers, and/or one or more additional antidegradants.

In some embodiments, the composition comprises from about 0.1 wt/wt % to about 10 wt/wt % of the Compound of the Disclosure, e.g., from about 0.1 wt/wt % to about 0.5 wt/wt %, from about 0.1 wt/wt % to about 1 wt/wt %, from about 0.1 wt/wt % to about 1.5 wt/wt %, from about 0.1 wt/wt % to about 2 wt/wt %, from about 0.1 wt/wt % to about 2.5 wt/wt %, from about 0.1 wt/wt % to about 3 wt/wt %, from about 0.1 wt/wt % to about 3.5 wt/wt %, from about 0.1 wt/wt % to about 4 wt/wt %, from about 0.1 wt/wt % to about 4.5 wt/wt %, from about 0.1 wt/wt % to about 5 wt/wt %, from about 0.1 wt/wt % to about 6 wt/wt %, from about 0.1 wt/wt % to about 7 wt/wt %, from about 0.1 wt/wt % to about 8 wt/wt %, from about 0.1 wt/wt % to about 9 wt/wt %, from about 0.1 wt/wt % to about 10 wt/wt %, from about 0.5 wt/wt % to about 1 wt/wt %, from about 0.5 wt/wt % to about 1.5 wt/wt %, from about 0.5 wt/wt % to about 2 wt/wt %, from about 0.5 wt/wt % to about 2.5 wt/wt %, from about 0.5 wt/wt % to about 3 wt/wt %, from about 0.5 wt/wt % to about 3.5 wt/wt %, from about 0.5 wt/wt % to about 4 wt/wt %, from about 0.5 wt/wt % to about 4.5 wt/wt %, from about 0.5 wt/wt % to about 5 wt/wt %, from about 0.5 wt/wt % to about 6 wt/wt %, from about 0.5 wt/wt % to about 7 wt/wt %, from about 0.5 wt/wt % to about 8 wt/wt %, from about 0.5 wt/wt % to about 9 wt/wt %, from about 0.5 wt/wt % to about 10 wt/wt %, from about 1 wt/wt % to about 2 wt/wt %, from about 1 wt/wt % to about 2.5 wt/wt %, from about 1 wt/wt % to about 3 wt/wt %, from about 1 wt/wt % to about 3.5 wt/wt %, from about 1 wt/wt % to about 4 wt/wt %, from about 1 wt/wt % to about 4.5 wt/wt %, from about 1 wt/wt % to about 5 wt/wt %, from about 1 wt/wt % to about 6 wt/wt %, from about 1 wt/wt % to about 7 wt/wt %, from about 1 wt/wt % to about 8 wt/wt %, from about 1 wt/wt % to about 9 wt/wt %, from about 1 wt/wt % to about 10 wt/wt %, from about 1.5 wt/wt % to about 2 wt/wt %, from about 1.5 wt/wt % to about 2.5 wt/wt %, from about 1.5 wt/wt % to about 3 wt/wt %, from about 1.5 wt/wt % to about 3.5 wt/wt %, from about 1.5 wt/wt % to about 4 wt/wt %, from about 1.5 wt/wt % to about 4.5 wt/wt %, from about 1.5 wt/wt % to about 5 wt/wt %, from about 1.5 wt/wt % to about 6 wt/wt %, from about 1.5 wt/wt % to about 7 wt/wt %, from about 1.5 wt/wt % to about 8 wt/wt %, from about 1.5 wt/wt % to about 9 wt/wt %, from about 1.5 wt/wt % to about 10 wt/wt %, from about 2 wt/wt % to about 2.5 wt/wt %, from about 2 wt/wt % to about 3 wt/wt %, from about 2 wt/wt % to about 3.5 wt/wt %, from about 2 wt/wt % to about 4 wt/wt %, from about 2 wt/wt % to about 4.5 wt/wt %, from about 2 wt/wt % to about 5 wt/wt %, from about 2 wt/wt % to about 6 wt/wt %, from about 2 wt/wt % to about 7 wt/wt %, from about 2 wt/wt % to about 8 wt/wt %, from about 2 wt/wt % to about 9 wt/wt %, from about 2 wt/wt % to about 10 wt/wt %, from about 2.5 wt/wt % to about 3 wt/wt %, from about 2.5 wt/wt % to about 3.5 wt/wt %, from about 2.5 wt/wt % to about 4 wt/wt %, from about 2.5 wt/wt % to about 4.5 wt/wt %, from about 2.5 wt/wt % to about 5 wt/wt %, from about 2.5 wt/wt % to about 6 wt/wt %, from about 2.5 wt/wt % to about 7 wt/wt %, from about 2.5 wt/wt % to about 8 wt/wt %, from about 2.5 wt/wt % to about 9 wt/wt %, from about 2.5 wt/wt % to about 10 wt/wt %, from about 3 wt/wt % to about 4 wt/wt %, from about 3 wt/wt % to about 4.5 wt/wt %, from about 3 wt/wt % to about 5 wt/wt %, from about 3 wt/wt % to about 6 wt/wt %, from about 3 wt/wt % to about 7 wt/wt %, from about 3 wt/wt % to about 8 wt/wt %, from about 3 wt/wt % to about 9 wt/wt %, from about 3 wt/wt % to about 10 wt/wt %, from about 3.5 wt/wt % to about 4 wt/wt %, from about 3.5 wt/wt % to about 4.5 wt/wt %, from about 3.5 wt/wt % to about 5 wt/wt %, from about 3.5 wt/wt % to about 6 wt/wt %, from about 3.5 wt/wt % to about 7 wt/wt %, from about 3.5 wt/wt % to about 8 wt/wt %, from about 3.5 wt/wt % to about 9 wt/wt %, from about 3.5 wt/wt % to about 10 wt/wt %, from about 4 wt/wt % to about 4.5 wt/wt %, from about 4 wt/wt % to about 5 wt/wt %, from about 4 wt/wt % to about 6 wt/wt %, from about 4 wt/wt % to about 7 wt/wt %, from about 4 wt/wt % to about 8 wt/wt %, from about 4 wt/wt % to about 9 wt/wt %, from about 4 wt/wt % to about 10 wt/wt %, from about 4.5 wt/wt % to about 5 wt/wt %, from about 4.5 wt/wt % to about 6 wt/wt %, from about 4.5 wt/wt % to about 7 wt/wt %, from about 4.5 wt/wt % to about 8 wt/wt %, from about 4.5 wt/wt % to about 9 wt/wt %, from about 4.5 wt/wt % to about 10 wt/wt %, from about 5 wt/wt % to about 6 wt/wt %, from about 5 wt/wt % to about 7 wt/wt %, from about 5 wt/wt % to about 8 wt/wt %, from about 5 wt/wt % to about 9 wt/wt %, from about 5 wt/wt % to about 10 wt/wt %, from about 6 wt/wt % to about 7 wt/wt %, from about 6 wt/wt % to about 8 wt/wt %, from about 6 wt/wt % to about 9 wt/wt %, from about 6 wt/wt % to about 10 wt/wt %, from about 7 wt/wt % to about 8 wt/wt %, from about 7 wt/wt % to about 9 wt/wt %, from about 8 wt/wt % to about 10 wt/wt %, from about 8 wt/wt % to about 9 wt/wt %, from about 8 wt/wt % to about 10 wt/wt %, or from about 9 wt/wt % to about 10 wt/wt % of a Compound of the Disclosure.

In some embodiments, a Composition of the Disclosure comprises from about 15 wt/wt % to about 85 wt/wt % of a Compound of the Disclosure. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of a Compound of the Disclosure.

In some embodiments, a Composition of the Disclosure comprises about 50 wt/wt % of a Compound of the Disclosure. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of a Compound of the Disclosure.

In another embodiment, a Composition of the Disclosure comprises a Compound of the Disclosure and one or more elastomers.

The term “elastomer” as used herein is a polymer with viscoelasticity (i.e., having both viscosity and elasticity) that typically has low intermolecular forces, low Young's modulus, and high failure strain. Elastomers can typically be cross-linked by heating in the presence of one or more cross-linking agents, a process called curing or vulcanization. Rubber is one type of elastomer. Non-limiting types of rubber include natural rubber (NR), synthetic rubber, and blends thereof. The term “natural rubber” as used herein refers to a naturally occurring elastomer that can be obtained from Hevea rubber trees. Non-limiting types of synthetic rubbers include unsaturated rubbers, saturated rubbers, rubbers with fluoro and fluoralkyl or fluoralkoxy substituent groups on the polymer chain (FKM), silicone rubbers (Q), and blends thereof. Non-limiting examples of unsaturated rubbers include polyisoprene rubber (IR), butyl rubber (IIR), polybutadiene rubber (BR), styrene-isoprene-butadiene rubber (SIBR), styrene butadiene rubber (SBR), nitrile butadiene rubber (NBR), chloroprene rubber (CR), ethylene propylene diene rubber (EPDM), and blends thereof. These unsaturated rubbers undergo cyclization and crosslinking reactions that lead to hardening of the aged part. As oxidation occurs, these vulcanizates harden and eventually become brittle products. Partial oxidation of vulcanizates leads to losses in performance when used in applications such as vehicle tire sidewalls. Saturated rubbers are rubbers that do not contain C═C unsaturation and include, but are not limited to, acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), rubbers with fluoro and fluoralkyl or fluoralkoxy substituent groups on the polymer chain (FKM), silicone rubber (Q), and blends thereof.

In some embodiments, the natural rubber comprises rubber derived from an alternative rubber plant. The term “natural rubber comprises rubber derived from an alternative rubber plant” as used herein refers to a naturally occurring elastomer that can be obtained from “non-Hevea” sources. In some embodiments, the alternative rubber plant is Parthenium argentatum (guayule) or Taraxacum kok-saghyz (Russian dandelion).

In some embodiments, the one or more elastomers further comprises recycled rubber. The term “recycled rubber” as used herein refers to an elastomer that has been reclaimed from scrap materials such as used tires.

In some embodiments, a Composition of the Disclosure comprises from about 15 wt/wt % to about 100 wt/wt % of one or more elastomers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 1 wt/wt % to about 100 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 5 wt/wt % to about 100 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 15 wt/wt % to about 100 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 25 wt/wt % to about 100 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 35 wt/wt % to about 100 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 45 wt/wt % to about 100 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 55 wt/wt % to about 100 wt/wt %, from about 65 wt/wt % to about 75 wt/wt %, from about 65 wt/wt % to about 85 wt/wt %, from about 65 wt/wt % to about 95 wt/wt %, from about 65 wt/wt % to about 100 wt/wt %, from about 75 wt/wt % to about 85 wt/wt %, from about 75 wt/wt % to about 95 wt/wt %, from about 75 wt/wt % to about 100 wt/wt %, from about 85 wt/wt % to about 95 wt/wt %, or from about 95 wt/wt % to about 100 wt/wt % of one or more elastomers.

In some embodiments, a Composition of the Disclosure comprises about 50 wt/wt % of one or more elastomers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, about 95 wt/wt %, or about 100 wt/wt % of one or more elastomers.

The term “phr” as used herein refers to parts per hundred parts of rubber by weight. The parts by weight of individual components are based on 100 parts by weight of the total mass of the one or more elastomers present in the composition.

In some embodiments, a Composition of the Disclosure comprises from about 1 phr to about 5 phr of a Compound of the Disclosure. In some embodiments, the composition comprises from about 0.01 phr to about 0.1 phr, from about 0.01 phr to about 0.5 phr, from about 0.01 phr to about 1 phr, from about 0.01 phr to about 2 phr, from about 0.01 phr to about 3 phr, from about 0.01 phr to about 4 phr, from about 0.01 phr to about 5 phr, from about 0.01 phr to about 7.5 phr, from about 0.01 phr to about 10 phr, from about 0.01 phr to about 20 phr, from about 0.1 phr to about 0.5 phr, from about 0.1 phr to about 1 phr, from about 0.1 phr to about 2 phr, from about 0.1 phr to about 3 phr, from about 0.1 phr to about 4 phr, from about 0.1 phr to about 5 phr, from about 0.1 phr to about 7.5 phr, from about 0.1 phr to about 10 phr, from about 0.1 phr to about 20 phr, from about 1 phr to about 2 phr, from about 1 phr to about 3 phr, from about 1 phr to about 4 phr, from about 1 phr to about 7.5 phr, from about 1 phr to about 10 phr, from about 1 phr to about 20 phr, from about 2 phr to about 3 phr, from about 2 phr to about 4 phr, from about 2 phr to about 5 phr, from about 2 phr to about 7.5 phr, from about 2 phr to about 10 phr, from about 2 phr to about 20 phr, from about 3 phr to about 4 phr, from about 3 phr to about 5 phr, from about 3 phr to about 7.5 phr, from about 3 phr to about 10 phr, from about 3 phr to about 20 phr, from about 4 phr to about 5 phr, from about 4 phr to about 7.5 phr, from about 4 phr to about 10 phr, from about 4 phr to about 20 phr, from about 5 phr to about 7.5 phr, from about 5 phr to about 10 phr, from about 5 phr to about 20 phr, from about 7.5 phr to about 10 phr, from about 7.5 phr to about 20 phr, or from about 10 phr to about 20 phr of a Compound of the Disclosure.

In some embodiments, a Composition of the Disclosure comprises about 3 phr of a Compound of the Disclosure. In some embodiments, the composition comprises about 0.01 phr, about 0.1 phr, about 0.5 phr, about 1 phr, about 2 phr, about 3 phr, about 4 phr, about 5 phr, about 7.5 phr, about 10 phr, or about 20 phr of a Compound of the Disclosure.

In some embodiments, a Composition of the Disclosure comprises a Compound of the Disclosure and one or more fillers.

The term “filler” as used herein is a substance that reinforces an elastomeric composition or gives an elastomeric composition other properties, including but not limited to expanding the volume of the composition. Non-limiting examples of fillers include carbon black, silica, kaolin, calcium silicate, talc, carbon nanotubes (CNT), carbon fibers (HCF), graphite, graphenes, aluminosilicates, starch, and fibers, and combinations thereof.

In some embodiments, the filler is derived from natural sources. For example, silica may be derived from rice husks.

In some embodiments, a Composition of the Disclosure comprises from about 15 wt/wt % to about 85 wt/wt % of one or more fillers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more fillers.

In some embodiments, a Composition of the Disclosure comprises about 50 wt/wt % of one or more fillers. In some embodiments, the composition comprises about 1 wt/wt %, about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more fillers.

In some embodiments, a Composition of the Disclosure comprises from about 30 phr to about 500 phr of one or more fillers. In some embodiments, the composition comprises from about 30 phr to about 50 phr, from about 30 phr to about 100 phr, from about 30 phr to about 150 phr, from about 30 phr to about 200 phr, from about 30 phr to about 250 phr, from about 30 phr to about 300 phr, from about 30 phr to about 350 phr, from about 30 phr to about 400 phr, from about 30 phr to about 450 phr, from about 30 phr to about 500 phr, from about 50 phr to about 100 phr, from about 50 phr to about 150 phr, from about 50 phr to about 200 phr, from about 50 phr to about 250 phr, from about 50 phr to about 300 phr, from about 50 phr to about 350 phr, from about 50 phr to about 400 phr, from about 50 phr to about 450 phr, from about 50 phr to about 500 phr, from about 100 phr to about 150 phr, from about 100 phr to about 200 phr, from about 100 phr to about 250 phr, from about 100 phr to about 300 phr, from about 100 phr to about 350 phr, from about 100 phr to about 400 phr, from about 100 phr to about 450 phr, from about 100 phr to about 500 phr, from about 150 phr to about 200 phr, from about 150 phr to about 250 phr, from about 150 phr to about 300 phr, from about 150 phr to about 350 phr, from about 150 phr to about 400 phr, from about 150 phr to about 450 phr, from about 150 phr to about 500 phr, from about 200 phr to about 250 phr, from about 200 phr to about 300 phr, from about 200 phr to about 350 phr, from about 200 phr to about 400 phr, from about 200 phr to about 450 phr, from about 200 phr to about 500 phr, from about 250 phr to about 300 phr, from about 250 phr to about 350 phr, from about 250 phr to about 400 phr, from about 250 phr to about 450 phr, from about 250 phr to about 500 phr, from about 300 phr to about 350 phr, from about 300 phr to about 400 phr, from about 300 phr to about 450 phr, from about 300 phr to about 500 phr, from about 350 phr to about 400 phr, from about 350 phr to about 450 phr, from about 350 phr to about 500 phr, from about 400 phr to about 450 phr, from about 400 phr to about 500 phr, or from about 450 phr to about 500 phr of one or more fillers.

In some embodiments, a Composition of the Disclosure comprises about 300 phr of one or more fillers. In some embodiments, the composition comprises about 30 phr, about 50 phr, about 100 phr, about 150 phr, about 200 phr, about 250 phr, about 350 phr, about 400 phr, about 450 phr, or about 500 phr of one or more fillers.

In some embodiments, a Composition of the Disclosure comprises a Compound of the Disclosure and one or more rubber chemicals. The term “rubber chemicals” as used herein refers to a compound or substance used to facilitate the vulcanization of rubber. Rubber chemicals include, but are not limited to, vulcanizing agents, accelerators, activators, and pre-vulcanization inhibitors.

The term “vulcanization” and the like as used herein refers to a process wherein cross-links are formed between elastomers to effect changes in the material properties of elastomers. In particular, vulcanization typically increases the rigidity and durability of elastomers. Vulcanization is carried out at room temperature or at elevated temperatures, depending on the nature of the elastomer(s), filler(s), and rubber chemical(s) being used. The term “curing” is also used in the art to describe this process.

The term “vulcanizing agent” as used herein refers to any substance that enables cross-linking between elastomers. Vulcanizing agents can enable cross-linking between separate polymer chains of an elastomer by various mechanisms, including, but not limited to, by formation of covalent bonds between the vulcanizing agent and two or more separate polymer chains or by generating radical species on separate polymer chains that can combine to form covalent bonds between the two polymer chains. Non-limiting examples of vulcanizing agents include sulfur, peroxides, vulcanized vegetable oil, factices and resins. Non-limiting examples of sulfur include octasulfur (S₈), cyclododecasulfur (S₁₂), and polymeric sulfur. Non-limiting examples of peroxides include benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy) hexane (DDPH), di-(2-tert-butylperoxyisopropyl)benzene (VC), butyl-4,4-di-(tert-butylperoxy) valerate (VAL), and 1,1-di(tert-butylperoxy)-3,3,5-trimethylcyclohexane (TMC). Nonlimiting examples of resins include bonding resins. The term “bonding resin” as used herein refers to a chemical such as resorcinol formaldehyde resins and phenolic resins that reacts with methylene donors (such as hexamethylenetetramine (HMTA) or hexamethoxymethyl melamine (HMMM)) to promote adhesion.

The term “accelerator” as used herein refers to any substance that increases the kinetics of vulcanization. In some embodiments, accelerators enable vulcanization to be performed at lower temperatures and/or to use the vulcanization agent, e.g., sulfur, more efficiently. Non-limiting examples of accelerators include guanidines, thiazoles, sulfenamides, thiurams, dithiocarbamates, xanthates, and thiophosphates. Non-limiting examples of guanidines include diphenylguanidine (DPG). Non-limiting examples of thiazoles include 2-mercaptobenzothiazole (MBT), zinc 2-mercaptobenzothiazole (ZMBT), mercaptobenzothiazole disulfide (MBTS), and N-tert-butyl-2-benzothiazole sulfenimide (TBSI). Non-limiting examples of sulfenamides include N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), and thiocarbamyl sulfenamide. Non-limiting examples of thiurams include dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), and tetramethylthiuram monosulfide (TMTM). Non-limiting examples of dithiocarbamates include zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), and zinc dibenzyldithiocarbamate (ZEBC).

The term “activator” as used herein refers to any substance that activates a vulcanizing agent and enables it to cross-link elastomers as described above. Activators may act via various mechanisms, including, but not limited to, by forming chemical complexes with accelerators or by coordinating to sulfur (when sulfur is used as a vulcanizing agent). Non-limiting examples of activators include metal oxides, acids, and metal complexex. Non-limiting examples of metal oxides include zinc oxide, magnesium oxide, and lead oxide. Non-limiting examples of acids include stearic acid and lauric acid. Non limiting examples of metal complexes include zinc ethylhexanoate.

The term “pre-vulcanization inhibitor” as used herein refers to compounds that delay the onset and/or the rate of vulcanization. These compounds are also referred to as “retarders.” Non-limiting examples of pre-vulcanization inhibitors include N-(cyclohexylthio) phthalimide (CTP), benzoic anhydride, salicylic anhydride, and phthalic anhydride.

In some embodiments, a Composition of the Disclosure comprises from about 15 wt/wt % to about 85 wt/wt % of one or more rubber chemicals. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more rubber chemicals.

In some embodiments, a Composition of the Disclosure comprises about 15 wt/wt % of one or more rubber chemicals. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more rubber chemicals.

In some embodiments, a Composition of the Disclosure comprises from about 1 phr to about 20 phr of one or more rubber chemicals. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more rubber chemicals.

In some embodiments, a Composition of the Disclosure comprises about 10 phr of one or more rubber chemicals. In some embodiments, the composition comprises about 0.1 phr, about 1 phr, about 5 phr, about 15 phr, about 20 phr, about 25 phr, about 30 phr, about 35 phr, or about 40 phr of one or more rubber chemicals.

The term “plasticizer” as used herein refers to a processing aid used to reduce the viscosity, increase the plasticity, and/or extend the volume of a composition. Plasticizers facilitate the process of mixing and forming a composition comprising an elastomer before the composition is vulcanized. Non-limiting examples of plasticizers include mineral oils (paraffinic, aromatic, or naphthenic), organic esters, resins, waxes, ester plasticizers, and naturally derived oils, such as soybean oil, vegetable oil, or orange oil.

In some embodiments, a Composition of the Disclosure comprises from about 15 wt/wt % to about 85 wt/wt % of one or more plasticizers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more plasticizers.

In some embodiments, a Composition of the Disclosure comprises about 15 wt/wt % of one or more plasticizers. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more plasticizers.

In some embodiments, a Composition of the Disclosure comprises from about 1 phr to about 20 phr of one or more plasticizers. In some embodiments, the composition comprises from about 0.1 phr to about 1 phr, from about 0.1 phr to about 5 phr, from about 0.1 phr to about 10 phr, from about 0.1 phr to about 15 phr, from about 0.1 phr to about 20 phr, from about 0.1 phr to about 25 phr, from about 0.1 phr to about 30 phr, from about 0.1 phr to about 35 phr, from about 0.1 phr to about 40 phr, from about 1 phr to about 5 phr, from about 1 phr to about 10 phr, from about 1 phr to about 15 phr, from about 1 phr to about 25 phr, from about 1 phr to about 30 phr, from about 1 phr to about 35 phr, from about 1 phr to about 40 phr, from about 5 phr to about 10 phr, from about 5 phr to about 15 phr, from about 5 phr to about 20 phr, from about 5 phr to about 25 phr, from about 5 phr to about 30 phr, from about 5 phr to about 35 phr, from about 5 phr to about 40 phr, from about 10 phr to about 15 phr, from about 10 phr to about 20 phr, from about 10 phr to about 25 phr, from about 10 phr to about 30 phr, from about 10 phr to about 35 phr, from about 10 phr to about 40 phr, from about 15 phr to about 20 phr, from about 15 phr to about 25 phr, from about 15 phr to about 30 phr, from about 15 phr to about 35 phr, from about 15 phr to about 40 phr, from about 20 phr to about 25 phr, from about 20 phr to about 30 phr, from about 20 phr to about 35 phr, from about 20 phr to about 40 phr, from about 25 phr to about 30 phr, from about 25 phr to about 35 phr, from about 25 phr to about 40 phr, from about 30 phr to about 35 phr, from about 30 phr to about 40 phr, or from about 35 phr to about 40 phr of one or more plasticizers.

In some embodiments, a Composition of the Disclosure comprises about 10 phr of one or more plasticizers. In some embodiments, the composition comprises about 0.1 phr, about 1 phr, about 5 phr, about 15 phr, about 20 phr, about 25 phr, about 30 phr, about 35 phr, or about 40 phr of one or more plasticizers.

In some embodiments, a Composition of the Disclosure further comprises one or more additional antidegradants that is (are) not a Compound of the Disclosure. In some embodiments, the one or more additional antidegradants is an antioxidant. In some embodiments, the one or more additional antidegradants is an antiozonant. In some embodiments, the one or more additional antidegradants is a heat stabilizer, a light stabilizer, or a fatigue crack stabilizer. Non-limiting examples of antidegradants include paraphenylenediamines (PPDs), trimethyl-dihydroquinolines (TMQs), phenolics or hindered phenolics, alkylated diphenylamines (DPAs), diphenylamine-ketone condensates, quinones, hydroquinones, amines, hindered amines, parafinic or microcrystalline waxes, and/or natural antidegradants. Non-limiting examples of PPDs include N¹-(4-methylpentan-2-yl)-N⁴-phenylbenzene-1,4-diamine (6PPD), N-(1,4-dimethylpentyl)-N′-phenyl-p-phenylenediamine (7PPD), N-sec-octyl-N′-phenyl-p-phenylenediamine (8PPD), N¹-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), and N—N′-dioctyl-p-phenylenediamine (88PD). Non-limiting examples of TMQs include 2,2,4-trimethyl-1,2-dihydroquinoline and oligomers or polymers thereof.

In some embodiments, a Composition of the Disclosure comprises from about 15 wt/wt % to about 85 wt/wt % of one or more additional antidegradants. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more additional antidegradants.

In some embodiments, a Composition of the Disclosure comprises about 15 wt/wt % of one or more additional antidegradants. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more additional antidegradants.

In some embodiments, a Composition of the Disclosure comprises from about 1 to about 5 phr of one or more additional antidegradants. In some embodiments, the composition comprises from about 0.001 phr to about 0.01 phr, from about 0.001 phr to about 0.1 phr, from about 0.001 phr to about 1 phr, from about 0.001 phr to about 5 phr, from about 0.001 phr to about 7.5 phr, from about 0.001 phr to about 10 phr, from about 0.01 phr to about 0.1 phr, from about 0.01 phr to about 1 phr, from about 0.01 phr to about 5 phr, from about 0.01 phr to about 7.5 phr, from about 0.01 phr to about 10 phr, from about 0.1 phr to about 1 phr, from about 0.1 phr to about 5 phr, from about 0.1 phr to about 7.5 phr, from about 0.1 phr to about 10 phr, from about 1 phr to about 7.5 phr, from about 1 phr to about 10 phr, from about 5 phr to about 7.5 phr, from about 5 phr to about 10 phr, or from about 7.5 phr to about 10 phr of one or more additional antidegradants.

In some embodiments, a Composition of the Disclosure comprises about 3 phr of one or more additional antidegradants. In some embodiments, the composition comprises about 0.001 phr, about 0.01 phr, about 0.1 phr, about 1 phr, about 2 phr, about 4 phr, about 5 phr, about 7.5 phr, or about 10 phr of one or more additional antidegradants.

In some embodiments, the disclosure provides a composition comprising a Compound of the Disclosure and one or more carriers. The term “carrier” as used herein refers to a solid that can adsorb a liquid while retaining the general properties of a solid at room temperature. In some embodiments, the carrier is an inert material. In some embodiments, the carrier has a high surface area. In some embodiments, the carrier comprises particles with diameters of less than 500 microns.

In some embodiments, the composition comprises from about 15 wt/wt % to about 85 wt/wt % of one or more carriers. In some embodiments, the composition comprises from about 1 wt/wt % to about 5 wt/wt %, from about 1 wt/wt % to about 15 wt/wt %, from about 1 wt/wt % to about 25 wt/wt %, from about 1 wt/wt % to about 35 wt/wt %, from about 1 wt/wt % to about 45 wt/wt %, from about 1 wt/wt % to about 55 wt/wt %, from about 1 wt/wt % to about 65 wt/wt %, from about 1 wt/wt % to about 75 wt/wt %, from about 1 wt/wt % to about 85 wt/wt %, from about 1 wt/wt % to about 95 wt/wt %, from about 5 wt/wt % to about 15 wt/wt %, from about 5 wt/wt % to about 25 wt/wt %, from about 5 wt/wt % to about 35 wt/wt %, from about 5 wt/wt % to about 45 wt/wt %, from about 5 wt/wt % to about 55 wt/wt %, from about 5 wt/wt % to about 65 wt/wt %, from about 5 wt/wt % to about 75 wt/wt %, from about 5 wt/wt % to about 85 wt/wt %, from about 5 wt/wt % to about 95 wt/wt %, from about 15 wt/wt % to about 25 wt/wt %, from about 15 wt/wt % to about 35 wt/wt %, from about 15 wt/wt % to about 45 wt/wt %, from about 15 wt/wt % to about 55 wt/wt %, from about 15 wt/wt % to about 65 wt/wt %, from about 15 wt/wt % to about 75 wt/wt %, from about 15 wt/wt % to about 95 wt/wt %, from about 25 wt/wt % to about 35 wt/wt %, from about 25 wt/wt % to about 45 wt/wt %, from about 25 wt/wt % to about 55 wt/wt %, from about 25 wt/wt % to about 65 wt/wt %, from about 25 wt/wt % to about 75 wt/wt %, from about 25 wt/wt % to about 85 wt/wt %, from about 25 wt/wt % to about 95 wt/wt %, from about 35 wt/wt % to about 45 wt/wt %, from about 35 wt/wt % to about 55 wt/wt %, from about 35 wt/wt % to about 65 wt/wt %, from about 35 wt/wt % to about 75 wt/wt %, from about 35 wt/wt % to about 85 wt/wt %, from about 35 wt/wt % to about 95 wt/wt %, from about 45 wt/wt % to about 55 wt/wt %, from about 45 wt/wt % to about 65 wt/wt %, from about 45 wt/wt % to about 75 wt/wt %, from about 45 wt/wt % to about 85 wt/wt %, from about 45 wt/wt % to about 95 wt/wt %, from about 55 wt/wt % to about 65 wt/wt %, from about 55 wt/wt % to about 75 wt/wt %, from about 55 wt/wt % to about 85 wt/wt %, from about 55 wt/wt % to about 95 wt/wt %, from about 65 wt/wt % to about 75 wt/wt %, from about 65 wt/wt % to about 85 wt/wt %, from about 65 wt/wt % to about 95 wt/wt %, from about 75 wt/wt % to about 85 wt/wt %, from about 75 wt/wt % to about 95 wt/wt %, or from about 85 wt/wt % to about 95 wt/wt % of one or more carriers.

In some embodiments, the composition comprises about 15 wt/wt % of one or more carriers. In some embodiments, the composition comprises about 1 wt/wt %, about 5 wt/wt %, about 10 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of one or more carriers.

The present disclosure also provides processes for preparing a composition comprising a Compound of the Disclosure and one or more carriers, the process comprising admixing the compound and the one or more carriers.

The present disclosure also provides lubricant compositions comprising a Compound of the Disclosure and a lubricant. Non-limiting examples of lubricants include mineral oil, higher molecular weight petroleum distillates such as aromatic, naphthenic, and paraffinic distillates, synthetic oils such as polyalpha-olefin (PAO), synthetic esters, polyalkylene glycols (PAG), phosphate esters, perfluoropolyether (PFPE), alkylated naphthlalenes (AN), silicate esters, ionic fluids, and multiply alkylated cyclopentanes (MAC), solid lubricants such as polytetrafluoroethylene (PTFE), graphite, hexagonal boron nitride, molybdenum disulfide, tungsten disulfide, aqueous lubricants such as hydrated brush polymers, and biolubricants such as triglyceride esters, high oleic canola oil, castor oil, palm oil, sunflower seed oil, and rapeseed oil.

The present disclosure also provides combustible fuel compositions comprising a combustible fuel and a Compound of the Disclosure. Non-limiting examples of combustible fuel include gasoline, diesel, kerosene, liquefied petroleum gas, synthetic fuel, and biodisesel.

The present disclosure also provides fuel additive compositions comprising a fuel additive and a Compound of the Disclosure. Non-limiting examples of fuel additives include oxygenates such as alcohols and ethers, antioxidants, stabilizers, detergents, antiknock agents, lead scavengers, fuel dyes, viscosity modifiers, and butyl rubber. In some embodiments, the butyl rubber is in the form of polyisobutylene succinimide. In some embodiments, the butyl rubber is added as a detergent to prevent fouling of diesel fuel injectors.

In some embodiments, the present disclosure provides a composition recited in Table 2.

TABLE 2

| Component | Component | Composition | Component | Component |
|---|---|---|---|---|
| 1 | 1 wt/wt % | Type | 2 | 2 wt/wt % |
| Compound | 0.1-10 | Tire or other | Elastomer | 5-95 |
| of the | | rubber article | | |
| Disclosure | | (belt, hose, | | |
| | | mount, etc) | | |
| | | (vulcanized) | | |
| Compound | 0.01-10 | In-tire | Elastomer | 5-95 |
| of the | | component or | | |
| Disclosure | | built up | | |
| | | component or | | |
| | | other end use | | |
| | | component | | |
| | | (tread, polymer | | |
| | | blends, etc) | | |
| | | (unvulcanized) | | |
| Compound | 5-95 | Masterbatch | Elastomer | 95-5 |
| of the | | | | |
| Disclosure | | | | |
| Compound | 5-95 | Premix | Filler | 95-5 |
| of the | | | | |
| Disclosure | | | | |
| Compound | 5-95 | Premix | Rubber | 95-5 |
| of the | | | chemical | |
| Disclosure | | | | |
| Compound | 5-95 | Premix | Plasticizer | 95-5 |
| of the | | | | |
| Disclosure | | | | |
| Compound | 5-95 | Premix | Additional | 95-5 |
| of the | | | anti- | |
| Disclosure | | | degradant(s) | |

In some embodiments, the vulcanized tire or other rubber article composition of Table 2 comprises about 0.1 wt/wt %, about 0.2 wt/wt %, about 0.3 wt/wt %, about 0.4 wt/wt %, about 0.5 wt/wt %, about 0.6 wt/wt %, about 0.7 wt/wt %, about 0.8 wt/wt %, about 0.9 wt/wt %, about 1 wt/wt %, about 2 wt/wt %, about 3 wt/wt %, about 4 wt/wt %, about 5 wt/wt %, about 6 wt/wt %, about 7 wt/wt %, about 8 wt/wt %, about 9 wt/wt %, or about 10 wt/wt % of a Compound of the Disclosure. In some embodiments, the vulcanized tire or other rubber article of Table 2 comprises comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one elastomer.

In some embodiments, the unvulcanized in-tire component or built up component or other end use component composition of Table 2 comprises about 0.01 wt/wt %, about 0.02 wt/wt %, about 0.03 wt/wt %, about 0.04 wt/wt %, about 0.05 wt/wt %, about 0.06 wt/wt %, about 0.07 wt/wt %, about 0.08 wt/wt %, about 0.09 wt/wt %, about 0.1 wt/wt %, about 0.2 wt/wt %, about 0.3 wt/wt %, about 0.4 wt/wt %, about 0.5 wt/wt %, about 0.6 wt/wt %, about 0.7 wt/wt %, about 0.8 wt/wt %, about 0.9 wt/wt %, about 1 wt/wt %, about 2 wt/wt %, about 3 wt/wt %, about 4 wt/wt %, about 5 wt/wt %, about 6 wt/wt %, about 7 wt/wt %, about 8 wt/wt %, about 9 wt/wt %, or about 10 wt/wt % of a Compound of the Disclosure. In some embodiments, the unvulcanized in-tire component or built up component or other end use component composition of Table 2 comprises comprises about 5 wt/wt %, about 10 wt/wt %, about 15 wt/wt %, about 20 wt/wt %, about 25 wt/wt %, about 30 wt/wt %, about 35 wt/wt %, about 40 wt/wt %, about 45 wt/wt %, about 50 wt/wt %, about 55 wt/wt %, about 60 wt/wt %, about 65 wt/wt %, about 70 wt/wt %, about 75 wt/wt %, about 80 wt/wt %, about 85 wt/wt %, about 90 wt/wt %, or about 95 wt/wt % of at least one elastomer.

### Vulcanized Elastomeric Articles

The present disclosure also provides vulcanized elastomeric articles comprising a Compound of the Disclosure. The term “vulcanized elastomeric article” refers to an article that is made by forming a composition comprising an elastomer into a specific shape and vulcanizing the composition to provide the article.

The present disclosure also provides vulcanized elastomeric articles prepared using a composition described herein.

In some embodiments, the vulcanized elastomeric article is prepared by admixing a Compound of the Disclosure, one or more elastomers, and, optionally, one or more fillers; one or more rubber chemicals; one or more plasticizers; and/or one or more additional antidegradants, and vulcanizing the mixture to give the vulcanized article.

In some embodiments, the vulcanized elastomeric article is a tire. In some embodiments, the tire is a passenger vehicle tire, a light truck tire, a heavy truck or bus tire, a motorcycle tire, an agriculture tire, an earthmover tire, an airplane tire, or a racing tire.

In some embodiments, the vulcanized elastomeric article is a component of a tire. In some embodiments, the component is a bead, a belt, a body ply, an inner liner, a sidewall, an undertread, or a tread.

In some embodiments, the vulcanized elastomeric article is a rubber overshoe, a sealing strip, an acoustic panel, an air spring, a bellow, a membrane, a tactile sensor, a crash pad, a hose, a conveyor belt, or a flooring.

### Vehicles

In some embodiments, the present disclosure also provides a vehicle comprising a vulcanized elastomeric article, e.g., a tire, wherein the vulcanized elastomeric article comprises a Compound of the Disclosure.

In some embodiments, the present disclosure also provides a vehicle comprising a vulcanized elastomeric article, e.g., a tire, wherein the vulcanized elastomeric article is prepared using a composition described herein.

In some embodiments, the vehicle is a passenger vehicle, a light truck, a heavy truck or bus, a motorcycle, a tractor, an earthmover, an airplane, or a race car.

### Processes

The present disclosure also provides processes for preparing a vulcanized elastomeric article, the process comprising:
(a) forming a composition described herein into a formed shape; and
(b) vulcanizing the formed shape,
to provide a vulcanized elastomeric article.

In some embodiments, the vulcanizing is performed at an average temperature of from about 140° C. to about 160° C. In some embodiments, the vulcanizing is performed at an average temperature of from about 80° C. to about 100° C., from about 80° C. to about 120° C., from about 80° C. to about 140° C., from about 80° C. to about 160° C., from about 80° C. to about 180° C., from about 80° C. to about 200° C., from about 100° C. to about 120° C., from about 100° C. to about 140° C., from about 100° C. to about 160° C., from about 100° C. to about 180° C., from about 100° C. to about 200° C., from about 120° C. to about 140° C., from about 120° C. to about 160° C., from about 120° C. to about 180° C., from about 120° C. to about 200° C., from about 140° C. to about 180° C., from about 140° C. to about 200° C., from about 160° C. to about 180° C., from about 160° C. to about 200° C., or from about 180° C. to about 200° C.

In some embodiments, the vulcanizing is performed at an average temperature of about 150° C. In some embodiments, the vulcanizing is performed at an average temperature of about 80° C., about 100° C., about 120° C., about 140° C., about 160° C., about 180° C., or about 200° C.

The present disclosure also provides processes for retreading tires, the process comprising:
(a) applying a composition described herein to a tire;
(b) disposing a pre-vulcanized tread around the tire;
(c) disposing a curing envelope around the tire; and
(d) vulcanizing the tire.

### Kits

The present disclosure also provides kits comprising a composition described herein, packaged in a manner, e.g., in a container, that facilitates use of the composition to practice the processes and/or methods of the present disclosure. In some embodiments, the kit comprises a composition described herein and instructions for using the composition in a vulcanizable elastomeric composition. In some embodiments, the kit comprises a composition described herein and instructions for using the composition to prepare a vulcanized elastomeric article. The composition may be packaged in any suitable container, such as a sealed bottle or vessel, with a label affixed to the ceontainer or included in the kit that describes the composition and proper use thereof.

### Particular Embodiments

The present disclosure also provides the following particular embodiments.

Embodiment 1. A compound having Formula (I):

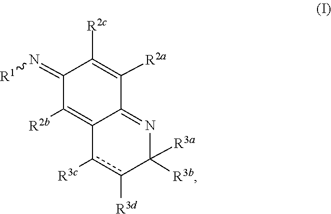

or a salt, solvate, or stereoisomer thereof, wherein:
   [symbol] is a single or a double bond;
   R¹ is selected from the group consisting of optionally substituted C₁-C₁₂ alkyl, —CHR^{1a}R^{1b}, C₃-C₆ cycloalkyl, 4- to 6-membered heterocyclyl, optionally substituted phenyl, and optionally substituted 5- or 6-membered heteroaryl;
   R^{1a} is selected from the group consisting of optionally substituted phenyl, C₃-C₆ cycloalkyl, and optionally substituted 5- or 6-membered heteroaryl;
   R^{1b} is selected from the group consisting of hydrogen and C₁-C₉ alkyl;
   R^{2a}, R^{2b}, and R^{2c} are independently selected from the group consisting of hydrogen, halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino; or
   R^{2a} and R^{2c} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
   R^{2b} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, optionally substituted 4- to 6-membered heterocyclyl, optionally substituted phenyl, optionally substituted 5- or 6-membered heteroaryl, aryloxy, arylthio, and arylamino;
   R^{3a} and R^{3b} are independently selected from the group consisting of hydrogen, halogen, C₁-C₉ alkyl, and optionally substituted phenyl; or
   R^{3a} and R^{3b} taken together with the carbon atom to which they are attached form a C₃-C₁₂ cycloalkyl;
   R^{3c} and R^{3d} are independently selected from the group consisting of hydrogen, halogen, C₁-C₉ alkyl, and optionally substituted phenyl; or
   R^{3c} and R^{3d} taken together with the two carbon atoms to which they are attached form a C₃-C₁₂ cycloalkyl;
   R⁴ is selected from the group consisting of hydrogen and C₁-C₉ alkyl; and
   R⁵ is selected from the group consisting of hydrogen and C₁-C₉ alkyl,
   with the proviso that the compound of Formula (I) is not:

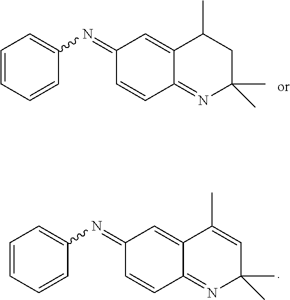 [SMILES: CC1CC(C)(C)N=C2C=CC(=Nc3ccccc3)C=C21 ; CC1=CC(C)(C)N=C2C=CC(=Nc3ccccc3)C=C12]

Embodiment 2. The compound of Embodiment 1, or a salt, solvate, or stereoisomer thereof, wherein R^{3a}, R^{3b}, and R^{3c} are independently selected from the group consisting of methyl, ethyl, propyl, iso-propyl, n-butyl, sec-butyl, iso-butyl, tert-butyl, pentyl, hexyl, heptyl, octyl, or nonyl.

Embodiment 3. The compound of Embodiment 2, or a salt, solvate, or stereoisomer thereof, wherein R^{3a}, R^{3b}, and R^{3c} are methyl.

Embodiment 4. The compound of any one of Embodiments 1-3, or a salt, solvate, or stereoisomer thereof, wherein R¹ is optionally substituted phenyl.

Embodiment 5. The compound of any one of Embodiments 1-3, or a salt, solvate, or stereoisomer thereof, wherein R¹ is C₁-C₁₂ alkyl.

Embodiment 6. The compound of any one of Embodiments 1-5, wherein R^{2a}, R^{2b}, and R^{2c} are hydrogen.

Embodiment 7. The compound of any one of Embodiments 1-5, or a salt, solvate, or stereoisomer thereof, having Formula (II):

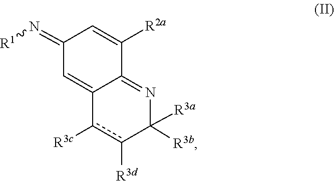

wherein R^{2a} is selected from the group consisting of halogen, C₁-C₉ alkyl, C₁-C₉ haloalkyl, C₃-C₈ cycloalkyl, C₁-C₈ alkoxy, C₁-C₈alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, and optionally substituted 5- or 6-membered heteroaryl.

Embodiment 8. The compound of Embodiment 7, or a salt, solvate, or stereoisomer thereof, wherein R^{2a} is C₁-C₉ alkyl.

Embodiment 9. The compound of Embodiment 8, or a salt, solvate, or stereoisomer thereof, wherein R^{2a} is methyl.

Embodiment 10. The compound of any one of Embodiments 1-5, or a salt, solvate, or stereoisomer thereof, having Formula (III):

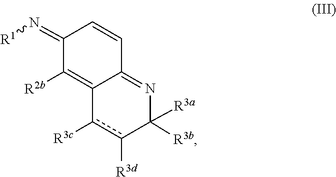

wherein R^{2b} is selected from the group consisting of halogen, C₁-C₉ alkyl, C₁-C₉ haloalkyl, C₃-C₈ cycloalkyl, C₁-C₈alkoxy, C₁-C₈alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

Embodiment 11. The compound of Embodiment 10, or a salt, solvate, or stereoisomer thereof, wherein R^{2b} is C₁-C₉ alkyl.

Embodiment 12. The compound of Embodiment 11, or a salt, solvate, or stereoisomer thereof, wherein R^{2b} is methyl.

Embodiment 13. The compound of any one of Embodiments 1-5, or a salt, solvate, or stereoisomer thereof, having Formula (IV):

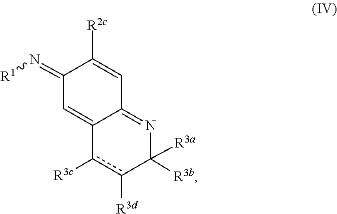

wherein R^{2c} is selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

Embodiment 14. The compound of Embodiment 13, or a salt, solvate, or stereoisomer thereof, wherein R^{2c} is C₁-C₉ alkyl.

Embodiment 15. The compound of Embodiment 14, or a salt, solvate, or stereoisomer thereof, wherein R^{2c} is methyl.

Embodiment 16. The compound of any one of Embodiments 1-5, or a salt, solvate, or stereoisomer thereof, having Formula (V):

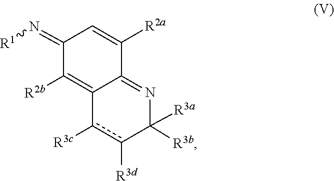

wherein R^{2a} and R^{2b} are independently selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —C(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

Embodiment 17. The compound of Embodiment 16, or a salt, solvate, or stereoisomer thereof, wherein R^{2a} and R^{2b} are independently C₁-C₉ alkyl.

Embodiment 18. The compound of Embodiment 17, or a salt, solvate, or stereoisomer thereof, wherein R^{2a} and R^{2b} are methyl.

Embodiment 19. The compound of any one of Embodiments 1-5, or a salt, solvate, or stereoisomer thereof, having Formula (VI):

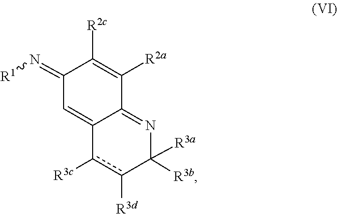

wherein R^{2a} and R^{2c} are independently selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

Embodiment 20. The compound of Embodiment 19, or a salt, solvate, or stereoisomer thereof, wherein R^{2a} and R^{2c} are independently C₁-C₉ alkyl.

Embodiment 21. The compound of Embodiment 20, or a salt, solvate, or stereoisomer thereof, wherein R^{2a} and R^{2c} are methyl.

Embodiment 22. The compound of any one of Embodiments 1-5, or a salt, solvate, or stereoisomer thereof, having Formula (VII):

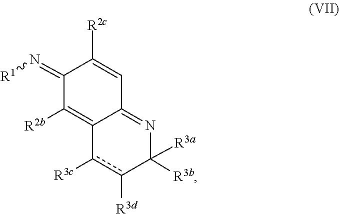

wherein R^{2b} and R^{2c} are independently selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

Embodiment 23. The compound of Embodiment 22, or a salt, solvate, or stereoisomer thereof, wherein R^{2b} and R^{2c} are independently C₁-C₉ alkyl.

Embodiment 24. The compound of Embodiment 23, or a salt, solvate, or stereoisomer thereof, wherein R^{2b} and R^{2c} are methyl.

Embodiment 25. The compound of any one of Embodiments 1-5, or a salt, solvate, or stereoisomer thereof, wherein R^{2a}, R^{2b}, and R^{2c} are independently selected from the group consisting of halogen, C₁-C₉ alkyl, C₁-C₆ alkoxy, and C₁-C₆ alkylthio.

Embodiment 26. The compound of any one of Embodiments 1-25, or a salt, solvate, or stereoisomer thereof, wherein [symbol] is a double bond.

Embodiment 27. The compound of any one of Embodiments 1-25, or a salt, solvate, or stereoisomer thereof, wherein [symbol] is a single bond.

### EXAMPLES

### Example 1

### Synthesis of 8-methoxy-2,2,4-trimethyl-N-phenylquinolin-6 (2H)-imine (Compound 7)

### Step 1

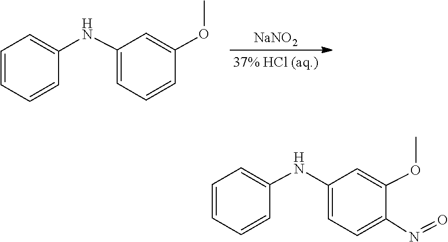

To a 250-mL round bottom flask fitted with a stir-bar and a thermometer was loaded 3-methoxydiphenylamine (11.8 g; 59.2 mmol) and glacial acetic acid (36 mL). The mixture was stirred under N₂ protection (the outlet was connected to a base trap containing about 250 mL of 1 M NaOH). Aq. 37% HCl (18 mL; 217 mmol HCl) was added by portions over a couple of minutes. 3-methoxydiphenylamine dissolved during the addition of aq. HCl. No exotherm was observed. A light-yellow solution was obtained. The flask was chilled with ice-water bath. Once the temperature reached 4-5° C., a solution of sodium nitrite (4.169 g; 60.4 mmol) in distilled water (12 mL) was gradually added over a period of 17-18 min such that the temperature was kept under 10° C. The resulting dark-red mixture was stirred between 2° C. and 9° C. for 30 min, then the chilling bath was removed. The reaction mixture was stirred without external cooling or heating (2-22° C.) for 2 h 40 min, whereupon a dark red solid separated. Distilled water (80 mL) was added and the slurry was stirred for a few minutes. The solid was collected by filtration under suction, then rinsed quickly with distilled water and EtOH (50 mL). Drying under vacuum at 40° C. for about 2 h, then at room temperature overnight, yielded 7.8 g (57% of the theoretical with respect to 3-methoxydiphenylamine) of a dark red powder. An aliquot of this material was characterized by LC-MS/UV (260 nm). The crude material was involved in the next step without further purification.

### Step 2

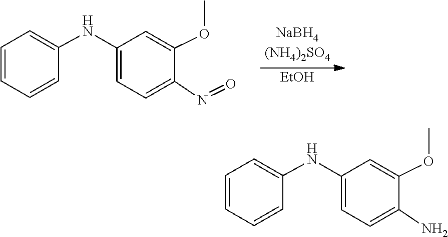 [SMILES: "EtOH" ; COc1cc(Nc2ccccc2)ccc1N=O ; COc1cc(Nc2ccccc2)ccc1N]

To a 500-mL flask fitted with a stir-bar and a thermometer were loaded 3-methoxy-4-nitroso-N-phenylaniline (See Step 1; 7.8 g; 34.2 mmol) and EtOH (210 mL). The mixture was stirred under N₂ protection (some solid remained insoluble; a red slurry was obtained). Ammonium sulfate (9.05 g; 68.5 mmol) was added in one portion (no exotherm was observed) and the mixture was stirred for approximately 1 h at room temperature. The flask was chilled with a cold tap water bath. Sodium borohydride (3.31 g; 87.5 mmol) was portion-wise added over a period of 16 min such that the temperature was kept under 28° C. (frothing occurred). The reaction mixture was stirred with the water bath for 110 min, during which the reaction mixture turned black. The water bath was removed. Approximately 10 min later, distilled water (150 mL) was slowly added and the mixture was stirred for a few minutes. The mixture was diluted with ethyl acetate (450 mL) in a separatory funnel containing distilled water (100 mL) and aq. 1 M NaOH (100 mL). After shaking and decanting, the aqueous layer was discarded. The organic layer was washed with aqueous 1 M NaOH (100 mL) and brine (100 mL), dried over MgSO₄, filtered, and stripped of volatiles under reduced pressure (rotary evaporator; water bath=55° C.). The resulting black oil was dried on a rotary evaporator (55° C.; <12 mbar) for 1 h. Crude yield=6.2 g as a black gooey material. The crude material was dissolved in toluene (10 mL). The obtained black solution was loaded into a 71 mm diameter chromatography column previously prepared with silica gel 60 (8″ height) and eluent (50% ethyl acetate in hexanes). The flask was rinsed with 50% ethyl acetate in hexanes (5-10 mL). The column was eluted with 50% ethyl acetate in hexanes. The portions (100 mL each) containing the desired product (TLC 50% ethyl acetate in hexanes; dots at Rf=0.7) were collected, combined, and stripped of volatiles under reduced pressure (rotary evaporator; water bath=50° C.). The resulting black/purple viscous liquid was dried on a rotary evaporator (55° C.; <2 mbar) for 15-30 min. Yield=4.3 g (58% based on 3-methoxy-4-nitroso-N-phenylaniline) as a dark viscous material.

### Step 3

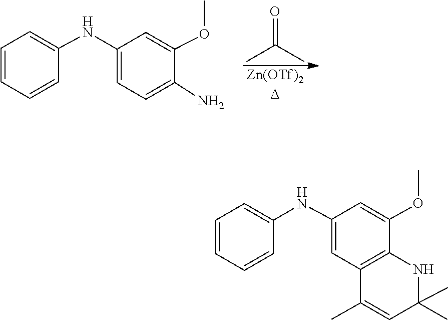

To a 250-mL flask fitted with a stir-bar and a reflux condenser were loaded 3-methoxy-N¹-phenylbenzene-1,4-diamine (4.3 g; 20.1 mmol), acetone (8.9 mL; 120 mol), and zinc (II) triflate (0.727 g; 2.00 mmol). The mixture was stirred under N₂ protection and heated (oil bath=62° C.) for 40.5 h. The substrate took about 2 h to initially dissolve, then a dark homogenous solution was obtained. Upon cooling to room temperature, the obtained black liquid was loaded into a 41 mm diameter chromatography column previously prepared with silica gel 60 (30 cm height) and eluent (20% ethyl acetate in hexanes). The flask was rinsed with additional acetone (3-4 mL). The column was eluted with 20% ethyl acetate in hexanes. Several fractions (approx. 30 mL each) were collected and characterized by silica gel TLC (20% ethyl acetate in hexanes). The fractions containing the dot at Rf=0.6 (desired product) were collected, combined, and stripped of volatiles under reduced pressure (rotary evaporator; water bath=50° C.). The resulting powder was dried on a rotary evaporator (55° C.; <3 mbars) for 20 min to give 8-methoxy-2,2,4-trimethyl-N-phenyl-1,2-dihydroquinolin-6-amine. Yield=2.5 g (42% of the theoretical with respect to 3-methoxy-N¹-phenylbenzene-1,4-diamine) as an almost-white crystalline powder.

### Step 4

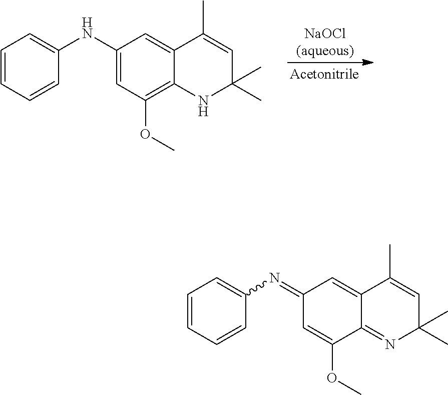 [SMILES: COc1cc(Nc2ccccc2)cc2c1NC(C)(C)C=C2C ; COC1=CC(=Nc2ccccc2)C=C2C(C)=CC(C)(C)N=C12]

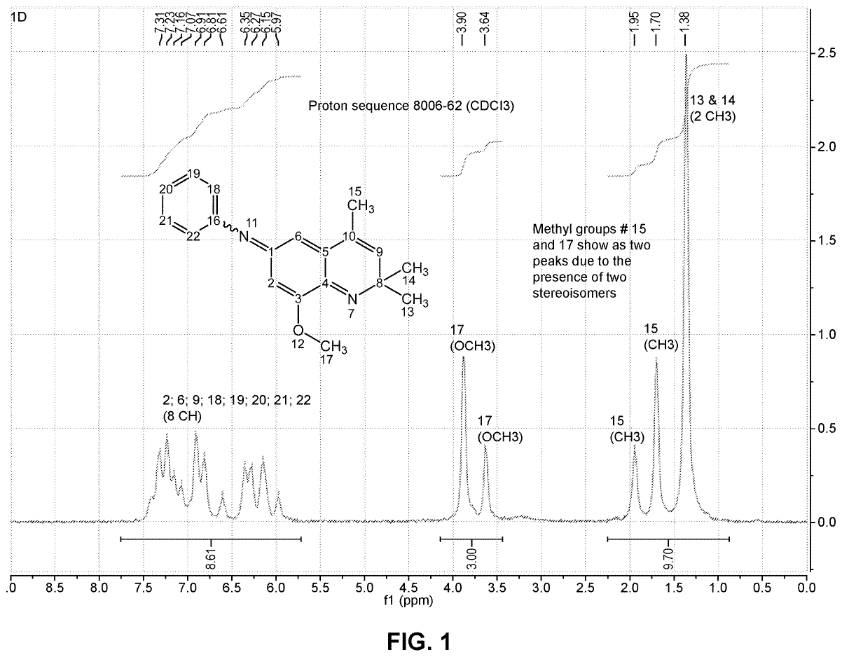

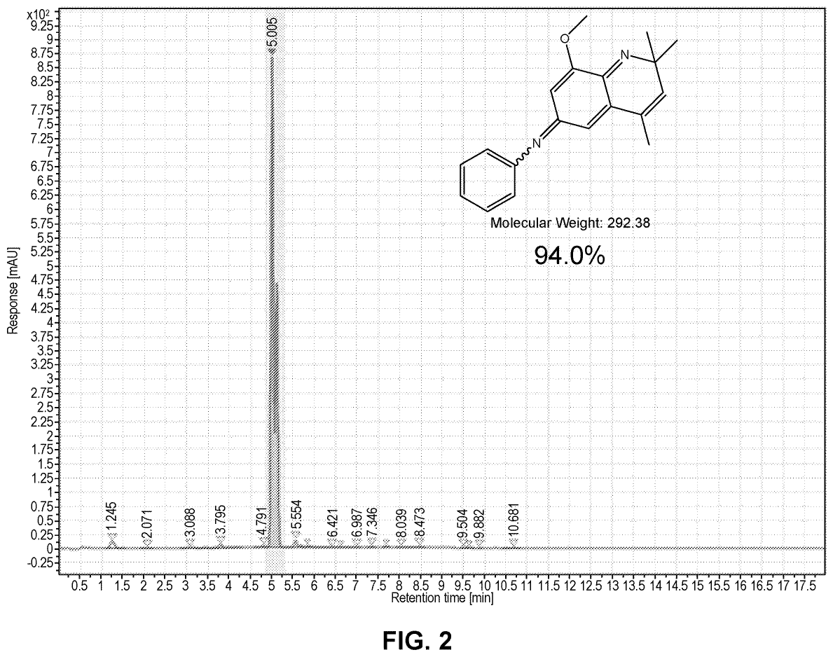

A 20-mL vial fitted with a stir-bar was loaded with the 8-methoxy-2,2,4-trimethyl-N-phenyl-1,2-dihydroquinolin-6-amine (300 mg; 1.02 mmol) and acetonitrile (5.5 mL). The mixture was stirred. The substrate did not dissolve completely. Aqueous 10-15 wt % NaOCI solution (0.65 mL) was added. The reaction mixture was stirred without external cooling or heating for 35 minutes whereupon an orange solution was obtained. An aqueous 17 wt % solution of sodium sulfite (1.0 mL; 1.35 mmol sulfite anion) was added to quench excess hypochlorite. Ethyl acetate (approx. 10-11 mL) and anhydrous magnesium sulfate was sequentially added under vigorous stirring. The mixture was gravimetrically filtered. The filtrate was stripped of its volatiles under reduced pressure (rotary evaporator; water bath=50° C.) to yield approx. 300 mg of Compound 7 as a dark solid. The structure was authenticated by 1H NMR spectroscopy, as shown in FIG. 1, as well as LC-MS, as shown in FIG. 2. The purity was 94.0 area % (combined areas of the two stereoisomers) as measured by LC-UV260 and shown in FIG. 2. The melting point of Compound 7 was measured to be in the range of 150-185° C.

### Example 2

### Oxidative Induction Time (OIT) Performance of Compound 7

The oxidative induction time (OIT) of selected Compounds of the Disclosure was evaluated. OIT is measured according to a procedure carried out in a differential scanning calorimeter (DSC; TA Instruments, Q200). In this procedure, a sample is held in a cell and heated under a nitrogen atmosphere to a preselected temperature. Oxygen (O₂) is then introduced to the cell and the length of time before the onset of degradation, as observed by the initiation of an endothermic process in the DSC trace, is measured.

0.5 wt % of a Compound of the Disclosure, N¹-(4-methylpentan-2-yl)-N⁴-phenylbenzene-1,4-diamine (6PPD), or no antidegradant (blank) was mixed with polyisoprene and heated isothermally at 150° C. and/or 160° C. in 02. The OIT in minutes is shown in Table 3. As indicated by the data in Table 3, Compounds of the Disclosure demonstrate antioxidant activity.

TABLE 3

| | OIT at 150° C. | OIT at 160° C. |
|---|---|---|
| Material | (minutes) | (minutes) |
| Blank | 2.0 | 1.2 |
| Compound 7 | 58.5 | 24.6 |
| 6PPD | 39.9 | 21.9 |

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

What is claimed is:

1. A compound having Formula (I): 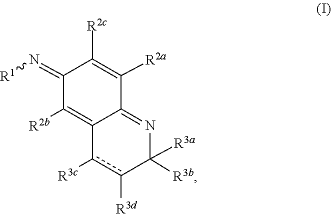
or a salt, solvate, or stereoisomer thereof, wherein: [symbol]
is a single or a double bond;
R¹ is selected from the group consisting of optionally substituted C₁-C₁₂ alkyl, —CHR^{1a}R^{1b}, C₃-C₆ cycloalkyl, 4- to 6-membered heterocyclyl, optionally substituted phenyl, and optionally substituted 5- or 6-membered heteroaryl;
R^{1a} is selected from the group consisting of optionally substituted phenyl, C₃-C₆ cycloalkyl, and optionally substituted 5- or 6-membered heteroaryl;
R^{1b} is selected from the group consisting of hydrogen and C₁-C₉ alkyl;
R^{2a}, R^{2b}, and R^{2c} are independently selected from the group consisting of hydrogen, halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino; or
R^{2a} and R^{2c} taken together with the two carbon atoms to which they are attached form an optionally substituted C₃-C₆ cycloalkyl, an optionally substituted 4- to 6-membered heterocyclyl, an optionally substituted 4- to 6-membered aryl, or an optionally substituted 4- to 6-membered heteroaryl; and
R^{2b} is selected from the group consisting of hydrogen, halogen, C₁-C₉ alkyl, C₁-C₉ haloalkyl, optionally substituted C₃-C₈ cycloalkyl, —OH, C₁-C₈ alkoxy, —SH, C₁-C₈ alkylthio, —C(═O)OR⁴, —OC(═O)R⁴, —C(═O)NR⁴R⁵, —NR⁴C(═O)R⁵, —NHR⁴, —NR⁴R⁵, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, optionally substituted 4- to 6-membered heterocyclyl, optionally substituted phenyl, optionally substituted 5- or 6-membered heteroaryl, aryloxy, arylthio, and arylamino;
R^{3a} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and 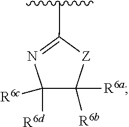
and
R^{3b} is selected from the group consisting of hydrogen, halogen, C₁-C₉ alkyl, and optionally substituted phenyl; or
R^{3a} and R^{3b} taken together with the carbon atom to which they are attached form a C₃-C₁₂ cycloalkyl;
R^{3c} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and 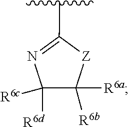
and
R^{3d} is selected from the group consisting of hydrogen, halogen, C₁-C₉ alkyl, and optionally substituted phenyl; or
R^{3c} and R^{3d} taken together with the two carbon atoms to which they are attached form a C₃-C₁₂ cycloalkyl;
R⁴ at each occurrence is independently selected from the group consisting of hydrogen and C₁-C₉ alkyl; and
R⁵ at each occurrence is independently selected from the group consisting of hydrogen and C₁-C₉ alkyl;
R^{6a}, R^{6b}, R^{6c}, and R^{6d} at each occurrence are independently selected from the group consisting of hydrogen, C₁-C₁₂ alkyl, and optionally substituted phenyl;
Z at each occurrence is independently selected from the group consisting of —O—, —S—, and —NR⁷—; and
R⁷ at each occurrence is independently selected from the group consisting of hydrogen and C₁-C₆ alkyl,
with the proviso that the compound of Formula (I) is not:

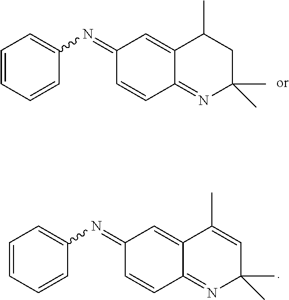 [SMILES: CC1CC(C)(C)N=C2C=CC(=Nc3ccccc3)C=C21 ; CC1=CC(C)(C)N=C2C=CC(=Nc3ccccc3)C=C12]

2. The compound of claim 1, or a salt, solvate, or stereoisomer thereof, wherein R^{3a}, R^{3b}, and R^{3c} are independently selected from the group consisting of methyl, ethyl, propyl, iso-propyl, n-butyl, sec-butyl, iso-butyl, tert-butyl, pentyl, hexyl, heptyl, octyl, or nonyl.

3. The compound of claim 2, or a salt, solvate, or stereoisomer thereof, wherein R^{3a}, R^{3b}, and R^{3c} are methyl.

4. The compound of any one of claims 1-3, or a salt, solvate, or stereoisomer thereof, wherein R¹ is optionally substituted phenyl.

5. The compound of any one of claims 1-3, or a salt, solvate, or stereoisomer thereof, wherein R¹ is C₁-C₁₂ alkyl.

6. The compound of any one of claims 1-5, wherein R^{2a}, R^{2b}, and R^{2c} are hydrogen.

7. The compound of any one of claims 1-5, or a salt, solvate, or stereoisomer thereof, having Formula (II): 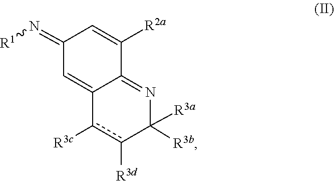
wherein R^{2a} is selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, and optionally substituted 5- or 6-membered heteroaryl.

8. The compound of claim 7, or a salt, solvate, or stereoisomer thereof, wherein R^{2a} is C₁-C₉ alkyl.

9. The compound of claim 8, or a salt, solvate, or stereoisomer thereof, wherein R^{2a} is methyl.

10. The compound of any one of claims 1-5, or a salt, solvate, or stereoisomer thereof, having Formula (III): 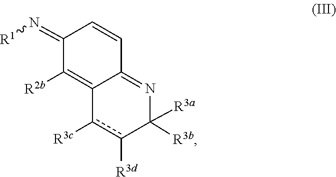
wherein R^{2b} is selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

11. The compound of claim 10, or a salt, solvate, or stereoisomer thereof, wherein R^{2b} is C₁-C₉ alkyl.

12. The compound of claim 11, or a salt, solvate, or stereoisomer thereof, wherein R^{2b} is methyl.

13. The compound of any one of claims 1-5, or a salt, solvate, or stereoisomer thereof, having Formula (IV): 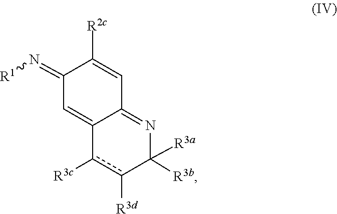
wherein R^{2c} is selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

14. The compound of claim 13, or a salt, solvate, or stereoisomer thereof, wherein R^{2c} is C₁-C₉ alkyl.

15. The compound of claim 14, or a salt, solvate, or stereoisomer thereof, wherein R^{2c} is methyl.

16. The compound of any one of claims 1-5, or a salt, solvate, or stereoisomer thereof, having Formula (V): 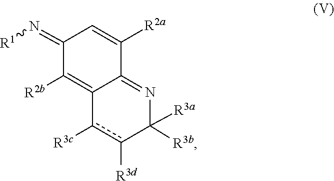
wherein R^{2a} and R^{2b} are independently selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

17. The compound of claim 16, or a salt, solvate, or stereoisomer thereof, wherein R^{2a} and R^{2b} are independently C₁-C₉ alkyl.

18. The compound of claim 17, or a salt, solvate, or stereoisomer thereof, wherein R^{2a} and R^{2b} are methyl.

19. The compound of any one of claims 1-5, or a salt, solvate, or stereoisomer thereof, having Formula (VI): 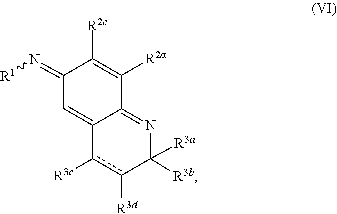
wherein R^{2a} and R^{2c} are independently selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

20. The compound of claim 19, or a salt, solvate, or stereoisomer thereof, wherein R^{2a} and R^{2c} are independently C₁-C₉ alkyl.

21. The compound of claim 20, or a salt, solvate, or stereoisomer thereof, wherein R^{2a} and R^{2c} are methyl.

22. The compound of any one of claims 1-5, or a salt, solvate, or stereoisomer thereof, having Formula (VII): 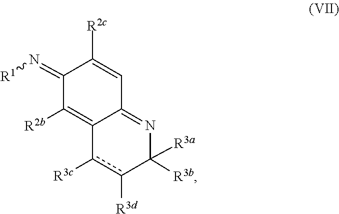
wherein R^{2b} and R^{2c} are independently selected from the group consisting of halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NHR⁴, —NR⁴R⁵, —OH, —SH, —SC(═O)R⁴, —SC(═O)SR⁴, —SC(═O)NHR⁴, —SC(═O)NR⁴R⁵, —NHC(═O)SR⁴, —NR⁴C(═O)SR⁵, —NHC(═S)SR⁴, —NR⁴C(═S)SR⁵, 4- to 6-membered heterocyclyl, optionally substituted phenyl, optionally substituted 5- or 6-membered heteroaryl, aryloxy, arylthio, and arylamino.

23. The compound of claim 22, or a salt, solvate, or stereoisomer thereof, wherein R^{2b} and R^{2c} are independently C₁-C₉ alkyl.

24. The compound of claim 23, or a salt, solvate, or stereoisomer thereof, wherein R^{2b} and R^{2c} are methyl.

25. The compound of any one of claims 1-5, or a salt, solvate, or stereoisomer thereof, wherein R^{2a}, R^{2b}, and R^{2c} are independently selected from the group consisting of halogen, C₁-C₉ alkyl, C₁-C₆ alkoxy, and C₁-C₆ alkylthio.

26. The compound of any one of claims 1-25, or a salt, solvate, or stereoisomer thereof, wherein [symbol] is a double bond.

27. The compound of any one of claims 1-25, or a salt, solvate, or stereoisomer thereof, wherein [symbol] is a single bond.

28. The compound of claim 1, or a salt, solvate, or stereoisomer thereof, wherein R^{3a} and R^{3c} are —C(═O)OR⁴.

29. The compound of claim 28, or a salt, solvate, or stereoisomer thereof, wherein each R⁴ is independently hydrogen or C₁-C₉ alkyl.

30. The compound of claim 29, or a salt, solvate, or stereoisomer thereof, wherein each R⁴ is ethyl.

31. The compound of any one of claims 28-30, or a salt, solvate, or stereoisomer thereof, wherein R^{3b} is C₁-C₉ alkyl.

32. The compound of claim 31, or a salt, solvate, or stereoisomer thereof, wherein R^{3b} is methyl.

33. The compound of claim 1, or a salt, solvate, or stereoisomer thereof, selected from any one or more of the compounds of Table 1A.

34. A compound having Formula (VIII): 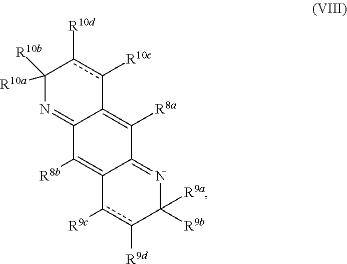
or a salt, solvate, or stereoisomer thereof, wherein:
each [symbol] is independently a single or a double bond;
R^{8a} and R^{8b} are independently selected from the group consisting of hydrogen, halogen, C₁-C₉ alkyl, C₁-C₉ haloalkyl, C₃-C₈ cycloalkyl, C₁-C₈ alkoxy, C₁-C₈ alkylthio, C(═O)OR⁴, —OC(═O)R⁴, —C(═O)NR⁴R⁵, —NR⁴C(═O)R⁵, —NR⁴R⁵, —OH, —SH, —SC(═O)R⁴, —SC(═O)SR⁴, —SC(═O)NR⁴R⁵, —NR⁴C(═O)SR⁵, —NR⁴C(═S)SR⁵, 4- to 6-membered heterocyclyl, optionally substituted phenyl, optionally substituted 5- or 6-membered heteroaryl, aryloxy, arylthio, and arylamino;
R^{9a} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and 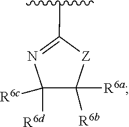
and
R^{6b} is selected from the group consisting of hydrogen, halogen, C₁-C₉ alkyl, and optionally substituted phenyl; or
R^{9a} and R^{6b} taken together with the carbon atom to which they are attached form a C₃-C₁₂ cycloalkyl;
R^{9c} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and 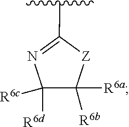
and
R^{6d} is selected from the group consisting of hydrogen, halogen, C₁-C₉ alkyl, and optionally substituted phenyl; or
R^{9c} and R^{6d} taken together with the two carbon atoms to which they are attached form a C₃-C₁₂ cycloalkyl;
R^{10a} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and 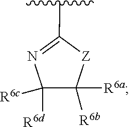
and
R^{10b} is selected from the group consisting of hydrogen, halogen, C₁-C₉ alkyl, and optionally substituted phenyl; or
R^{10a} and R^{10b} taken together with the carbon atom to which they are attached form a C₃-C₁₂ cycloalkyl;
R^{10c} is selected from the group consisting of hydrogen, halogen, C₁-C₉ alkyl, optionally substituted phenyl, —C(═O)OR⁴, —C(═O)NR⁴R⁵, —C(═O)SR⁴, —CH₂OR⁴, —CH₂OC(═O)R⁴, and 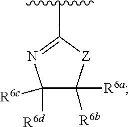
and
R^{10d} is selected from the group consisting of hydrogen, halogen, C₁-C₉ alkyl, and optionally substituted phenyl; or
R^{10c} and R^{10d} taken together with the two carbon atoms to which they are attached form a C₃-C₁₂ cycloalkyl;
Z at each occurrence is independently selected from the group consisting of —O—, —S—, and —NR⁷—;
R⁴ at each occurrence is independently selected from the group consisting of hydrogen, C₁-C₉ alkyl, C₃-C₆ cycloalkyl, and optionally substituted phenyl; and
R⁵ at each occurrence is independently selected from the group consisting of hydrogen, C₁-C₉ alkyl, C₃-C₆ cycloalkyl, and optionally substituted phenyl;
R^{6a}, R^{6b}, R^{6c}, and R^{6d} at each occurrence is independently are independently selected from the group consisting of hydrogen, C₁-C₁₂ alkyl, and optionally substituted phenyl; and
R⁷ at each occurrence is independently selected from the group consisting or hydrogen and C₁-C₆ alkyl.

35. The compound of claim 34, or a salt, solvate, or stereoisomer thereof, having Formula (IX):

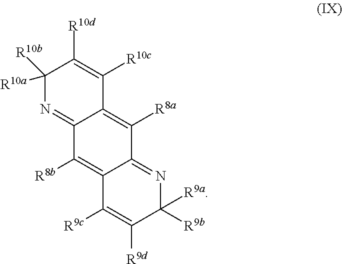

36. The compound of claim 34, or a salt, solvate, or stereoisomer thereof, having Formula (X):

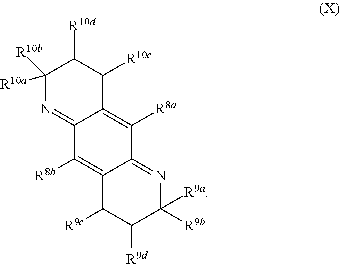

37. The compound of any one of claims 34-36, or a salt, solvate, or stereoisomer thereof, wherein R^{8a} and R^{8b} are hydrogen.

38. The compound of any one of claims 34-37, or a salt, solvate, or stereoisomer thereof, wherein R^{9a}, R^{9b}, and R^{9c} are independently selected from the group consisting of methyl, ethyl, propyl, iso-propyl, n-butyl, sec-butyl, iso-butyl, tert-butyl, pentyl, hexyl, heptyl, octyl, and nonyl.

39. The compound of claim 38, or a salt, solvate, or stereoisomer thereof, wherein R^{9a}, R^{9b}, and R^{9c} are methyl, ethyl, or iso-butyl.

40. The compound of claim 39, or a salt, solvate, or stereoisomer thereof, wherein R^{9a} and R^{9c} are methyl.

41. The compound of claim 39, or a salt, solvate, or stereoisomer thereof, wherein R^{9a} and R^{9c} are ethyl.

42. The compound of claim 39, or a salt, solvate, or stereoisomer thereof, wherein R^{9a} and R^{9c} are iso-butyl.

43. The compound of any one of claims 34-37, or a salt, solvate, or stereoisomer thereof, wherein R^{9a} and R^{9c} are —C(═O)OR⁴, —C(═O)NR⁴R⁵, —C(═O)SR⁴, —CH₂OR⁴, —CH₂OC(═O)R⁴, or

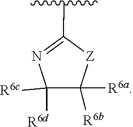

44. The compound of claim 43, or a salt, solvate, or stereoisomer thereof, wherein R^{9a} and R^{9c} are —C(═O)OR⁴.

45. The compound of claim 44, or a salt, solvate, or stereoisomer thereof, wherein each R⁴ is hydrogen, methyl or ethyl.

46. The compound of claim 45, or a salt, solvate, or stereoisomer thereof, wherein each R⁴ is ethyl.

47. The compound of any one of claims 43-46, or a salt, solvate, or stereoisomer thereof, wherein R^{6b} is methyl.

48. The compound of any one of claims 34-47, or a salt, solvate, or stereoisomer thereof, wherein R^{6d} is hydrogen.

49. The compound of any one of claims 34-48, or a salt, solvate, or stereoisomer thereof, wherein R^{10a}, R^{10b}, and R^{10c} are independently selected from the group consisting of methyl, ethyl, propyl, iso-propyl, n-butyl, sec-butyl, iso-butyl, tert-butyl, pentyl, hexyl, heptyl, octyl, and nonyl.

50. The compound of claim 49, or a salt, solvate, or stereoisomer thereof, wherein R^{10a}, R^{10b} and R^{10c} are methyl, ethyl, or iso-butyl.

51. The compound of claim 50, or a salt, solvate, or stereoisomer thereof, wherein R^{10a} and R^{10c} are methyl.

52. The compound of claim 51, or a salt, solvate, or stereoisomer thereof, wherein R^{10a} and R^{10c} are ethyl.

53. The compound of claim 51, or a salt, solvate, or stereoisomer thereof, wherein R^{10a} and R^{10c} are iso-butyl.

54. The compound of any one of claims 34-53, or a salt, solvate, or stereoisomer thereof, wherein R^{10a} and R^{10c} are —C(═O)OR⁴, —C(═O)NR⁴R⁵, —C(═O)SR⁴, —CH₂OR⁴, —CH₂OC(═O)R⁴, or

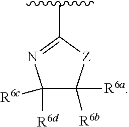

55. The compound of claim 54, or a salt, solvate, or stereoisomer thereof, wherein R^{10a} and R^{10c} are —C(═O)OR⁴.

56. The compound of claim 55, or a salt, solvate, or stereoisomer thereof, wherein each R⁴ is hydrogen, methyl or ethyl.

57. The compound of any one of claims 34-56, or a salt, solvate, or stereoisomer thereof, wherein R^{10b} is methyl.

58. The compound of any one of claims 34-57, or a salt, solvate, or stereoisomer thereof, wherein R^{10d} is hydrogen.

59. The compound of any one of claims 34-58, or a salt, solvate, or stereoisomer thereof, wherein R^{9a} and R^{10a} are the same.

60. The compound of any one of claims 34-59, or a salt, solvate, or stereoisomer thereof, wherein R^{9b} and R^{10b} are the same.

61. The compound of any one of claims 34-60, or a salt, solvate, or stereoisomer thereof, wherein R^{9c} and R^{10c} are the same.

62. The compound of any one of claims 34-61, or a salt, solvate, or stereoisomer thereof, wherein R^{6d} and R^{10d} are the same.

63. The compound of claim 34, or a salt, solvate, or stereoisomer thereof, selected from any one or more of the compounds of Table 1B.

64. A composition comprising:
(i) the compound of any one of claims 1-63; and
(ii) one or more elastomers; or
(iii) one or more fillers; or
(iv) one or more rubber chemicals; or
(v) one or more plasticizers; or
(vi) one or more additional antidegradants; or
(vii) a combination of one or more elastomers, one or more fillers, one or more rubber chemicals, one or more plasticizers, and/or one or more additional antidegradants.

65. The composition of claim 64, wherein the composition comprises one or more elastomers.

66. The composition of claim 64 or 65, wherein the composition comprises from about wherein the composition comprises from about 0.1 wt/wt % to about 10 wt/wt % of the compound.

67. The composition of any one of claims 64-66, wherein the one or more elastomers comprise natural rubber (NR).

68. The composition of claim 67, wherein the one or more elastomers comprises from about 5 wt/wt % to about 80 wt/wt % natural rubber (NR).

69. The composition of claim 67 or 68, wherein the natural rubber comprises rubber derived from an alternative rubber plant.

70. The composition of claim 69, wherein the alternative rubber plant is Parthenium argentatum (guayule) or Taraxacum kok-saghyz (Russian dandelion).

71. The composition of any one of claims 65-70, wherein the one or more elastomers comprise synthetic rubber.

72. The composition of claim 71, wherein the synthetic rubber comprises an unsaturated rubber, a saturated rubber, a rubber with fluoro and fluoralkyl or fluoralkoxy substituent groups on the polymer chain (FKM), a silicone rubber (Q), or a blend thereof.

73. The composition of claim 72, wherein the unsaturated rubber comprises polyisoprene rubber (IR), butyl rubber (IIR), polybutadiene rubber (BR), styrene butadiene rubber (SBR), nitrile butadiene rubber (NBR), chloroprene rubber (CR), ethylene propylene diene rubber (EPDM), or a blend thereof.

74. The composition of claim 73, wherein the unsaturated rubber comprises styrene butadiene rubber (SBR).

75. The composition of claim 74, wherein the one or more elastomers comprise from about 5 wt/wt % to about 80 wt/wt % styrene butadiene rubber (SBR).

76. The composition of any one of claims 73-75, wherein the unsaturated rubber comprises polybutadiene rubber (BR).

77. The composition of claim 76, wherein the one or more elastomers comprise from about 5 wt/wt % to about 80 wt/wt % polybutadiene rubber (BR).

78. The composition of any one of claims 73-77, wherein the unsaturated rubber comprises butyl rubber (IIR).

79. The composition of claim 78, wherein the one or more elastomers comprise from about 1 wt/wt % to about 30 wt/wt % butyl rubber (IIR).

80. The composition of any one of claims 72-79, wherein the saturated rubber comprises acrylic rubber (ACM), chlorinated polyethylene (CM), chlorosulfonated polyethylene (CSM), polychloromethyloxiran (CO), ethylene-ethyl acrylate copolymer (EAM), epichlorohydrin rubber (ECO), ethylene propylene rubber (EPM), ethylenevinylacetate copolymer (EVM), or a blend thereof.

81. The composition of claims any one of claims 64-80, wherein the one or more elastomers further comprises recycled rubber.

82. The composition of any one of claims 64-81, wherein the composition comprises from about 0.1 phr to about 10 phr of the compound.

83. The composition of claim 82, wherein the composition comprises from about 0.5 phr to about 5 phr of the compound.

84. The compound of claim 83, wherein the composition comprises from about 1 phr to about 5 phr of the compound.

85. The composition of any one of claims 64-84, wherein the composition comprises one or more fillers.

86. The composition of claim 85, wherein the composition comprises from about 15 wt/wt % to about 85 wt/wt % of the compound and from about 15 wt/wt % to about 85 wt/wt % of the one or more fillers.

87. The composition of claim 85 or 86, wherein the composition comprises from about 30 phr to about 500 phr of one or more fillers.

88. The composition of any one of claims 85-87, wherein the one or more fillers comprise carbon black, silica, kaolin, calcium silicate, talc, carbon nanotubes (CNT), carbon fibers (HCF), graphite, graphenes, aluminosilicates, starch, fibers, or a combination thereof.

89. The composition of claim 88, wherein the one or more fillers comprise silica.

90. The composition of claim 89, wherein the silica is derived from rice husks.

91. The composition of any one of claims 85-90, wherein the one or more fillers comprise carbon black.

92. The composition of any one of claims 64-63, wherein the composition comprises one or more rubber chemicals.

93. The composition of claim 92, wherein the composition comprises from about 15 wt/wt % to about 85 wt/wt % of the compound and from about 15 wt/wt % to about 85 wt/wt % of one or more rubber chemicals.

94. The composition of claim 92 or 93, wherein the composition comprises from about 0.1 phr to about 30 phr of one or more rubber chemicals.

95. The composition of claim 94, wherein the composition comprises from about 1 phr to about 20 phr of one or more rubber chemicals.

96. The composition of any one of claims 92-95, wherein the one or more rubber chemicals comprise one or more vulcanizing agents, one or more accelerators, one or more activators, one or more pre-vulcanization inhibitors, or a combination thereof.

97. The composition of claim 96, wherein the one or more rubber chemicals comprise one or more vulcanizing agents.

98. The composition of claim 97, wherein the one or more vulcanizing agents comprise sulfur, peroxide, resin, or a combination thereof.

99. The composition of claim 98, wherein the sulfur is octasulfur (S₈), cyclododecasulfur (S₁₂), polymeric sulfur, or a combination thereof.

100. The composition of claim 98, wherein the peroxide is benzoyl peroxide, dicumyl peroxide (DC), 2,5-dimethyl-2,5-di-(tert-butylperoxy)-3-hexyne (2,5 Tri), 2,5-dimethyl-2,5-di(tert-butylperoxy) hexane (DDPH), di-(2-tert-butylperoxyisopropyl)benzene (VC), butyl-4,4-di-(tert-butylperoxy) valerate (VAL), 1,1-di(tert-butylperoxy)-3,3,5-trimethylcyclohexane (TMC), or a combination thereof.

101. The composition of claim 98, wherein the resin is a bonding resin.

102. The composition of claims 96-101, wherein the one or more rubber chemicals comprise one or more accelerators.

103. The composition of claim 102, wherein the one or more accelerators comprise a guanidine, a thiazole, a sulfenamide, a thiuram, a dithiocarbamate, a xanthate, a thiophosphate, or a combination thereof.

104. The composition of claim 103, wherein the guanidine is diphenylguanidine (DPG).

105. The composition of claim 103, wherein the thiazole comprises 2-mercaptobenzothiazole (MBT), zinc 2-mercaptobenzothiazole (ZMBT), mercaptobenzothiazole disulfide (MBTS), N-tert-butyl-2-benzothiazole sulfenimide (TBSI), or a combination thereof.

106. The composition of claim 103, wherein the sulfenamide comprises N-tert-butyl-2-benzothiazylsulfenamide (TBBS), N-cyclohexylbenzothiazol-2-sulfenamide (CBS), dicyclohexyl-2-benzothiazolesulfenamide (DCBS), N-oxydiethylene benzothiazole sulfenamide (OBTS), N-oxydiethylenethiocarbamyl-N′-oxydiethylene sulfenamide (OTOS), thiocarbamyl sulfenamide, or a combination thereof.

107. The composition of claim 103, wherein the thiuram is dimethylcarbamothioic dithioperoxyanhydride (thiram), dipentamethylene thiuram tetrasulfide (DPIT), tetrabenzyl thiuram disulfide (TBzTD), tetraethylthiuram disulfide (TETD), tetramethylthiuram disulfide (TMTD), tetramethylthiuram monosulfide (TMTM), or a combination thereof.

108. The composition of claim 103, wherein the dithiocarbamate comprises zinc dimethyldithiocarbamate (ZDMC), zinc diethyldithiocarbamate (ZDEC), zinc dibutyldithiocarbamate (ZDBC), nickel dibutyldithiocarbamate (NDBC), sodium dibenzyldithiocarbamate (SBEC), sodium diethyldithiocarbamate (SDEC), tellurium diethyldithiocarbamate (TDEC), zinc dibenzyldithiocarbamate (ZEBC), or a combination thereof.

109. The composition of any one of claims 96-108, wherein the one or more rubber chemicals comprise one or more activators.

110. The composition of claim 109, wherein the one or more activators comprise a metal oxide, an acid, a metal complex, or a combination thereof.

111. The composition of claim 110, wherein the metal oxide comprises zinc oxide, magnesium oxide, lead oxide, or a combination thereof.

112. The composition of claim 110, wherein the acid comprises stearic acid, lauric acid, or a combination thereof.

113. The composition of claim 110, wherein the metal complex comprises zinc ethylhexanoate.

114. The composition of any one of claims 96-113, wherein the one or more rubber chemicals comprise one or more pre-vulcanization inhibitors.

115. The composition of claim 114, wherein the one or more pre-vulcanization inhibitors comprise N-(cyclohexylthio) phthalimide (CTP), benzoic anhydride, salicylic anhydride, phthalic anhydride, or a combination thereof.

116. The composition of any one of claims 64-115, wherein the composition comprises one or more plasticizers.

117. The composition of claim 116, wherein the composition comprises from about 15 wt/wt % to about 85 wt/wt % of the compound and from about 15 wt/wt % to about 85 wt/wt % of one or more plasticizers.

118. The composition of claim 116 or 117, wherein the composition comprises from about 0.1 phr to about 30 phr of one or more plasticizers.

119. The composition of claim 118, wherein the composition comprises from about 1 phr to about 20 phr of one or more plasticizers.

120. The composition of any one of claims 116-119, wherein the one or more plasticizers comprise a mineral oil, an organic ester, a resin, a wax, an ester plasticizer, a naturally derived oil, or a combination thereof.

121. The composition of claim 120, wherein the mineral oil is naphthenic oil, paraffinic oil, or aromatic oil.

122. The composition of claim 120, wherein the naturally derived oil is soybean oil, vegetable oil, or orange oil.

123. The composition of any one of claims 64-122, wherein the composition further comprises one or more additional antidegradants.

124. The composition of claim 123, wherein the composition comprises from about 15 wt/wt % to about 85 wt/wt % of the compound and from about 15 wt/wt % to about 85 wt/wt % of the one or more additional antidegradants.

125. The composition of claim 123 or 124, wherein the one or more additional antidegradants is present in an amount of from about 0.001 phr to about 10 phr.

126. The composition of claim 125, wherein the one or more additional antidegradants is present in an amount of from about 0.1 phr to about 5 phr.

127. The composition of claim 126, wherein the one or more additional antidegradants is present in an amount of from about 0.5 phr to about 5 phr.

128. The composition of claim 127, wherein the one or more additional antidegradants is present in an amount of from about 1 phr to about 5 phr.

129. The composition of any one of claims 123-128, wherein the one or more additional antidegradants is an antioxidant.

130. The composition of any one of claims 123-128, wherein the one or more additional antidegradants is an antiozonant.

131. The composition of any one of claims 123-130, wherein the one or more additional antidegradants is a paraphenylenediamine (PPD), a trimethyldihydroquinoline (TMQ), a phenolic, an alkylated diphenylamine (DPA), or a diphenylamine-ketone condensate.

132. The composition of claim 131, wherein the PPD is N¹-(4-methylpentan-2-yl)-N⁴-phenylbenzene-1,4-diamine (6PPD), N-(1,4-dimethylpentyl)-N′-phenyl-p-phenylenediamine (7PPD), N-sec-octyl-N′-phenyl-p-phenylenediamine (8PPD), N¹-phenyl-N⁴-(propan-2-yl)benzene-1,4-diamine (IPPD), N,N′-di-sec-butyl-p-phenylenediamine (44PD), N,N′-bis(1,3-dimethylbutyl)-p-phenylenediamine (66PD), N,N′-bis(1,4-dimethylpentyl)-p-phenylenediamine (77PD), or N—N′-dioctyl-p-phenylenediamine (88PD).

133. The composition of claim 132, wherein the PPD is 6PPD.

134. The composition of claim 131, wherein the TMQ is 2,2,4-trimethyl-1,2-dihydroquinoline or an oligomer or polymer thereof.

135. A composition comprising the compound of any one of claims 1-63 and one or more carriers.

136. A process for preparing the composition of claim 135, the process comprising admixing the compound and the one or more carriers.

137. A vulcanized elastomeric article comprising the compound of any one of claims 1-63.

138. A vulcanized elastomeric article prepared using the composition of any one of claims 104-135.

139. The vulcanized elastomeric article of claim 137 or 138, wherein the vulcanized elastomeric article is a tire.

140. The vulcanized elastomeric article of claim 139, wherein the tire is a passenger vehicle tire, a light truck tire, a heavy truck or bus tire, a motorcycle tire, an agriculture tire, an earthmover tire, an airplane tire, or a racing tire.

141. The vulcanized elastomeric article of claim 137 or 138, wherein the vulcanized elastomeric article is a component of a tire.

142. The vulcanized elastomeric article of claim 141, wherein the component is a bead, a belt, a body ply, an inner liner, a sidewall, an undertread, or a tread.

143. The vulcanized elastomeric article of claim 136 or 137, wherein the vulcanized elastomeric article is a rubber overshoe, a sealing strip, an acoustic panel, an air spring, a bellow, a membrane, a tactile sensor, a crash pad, a hose, a conveyor belt, or a flooring.

144. A vehicle comprising the vulcanized elastomeric article of any one of claims 137-143.

145. A process for preparing a vulcanized elastomeric article, the process comprising:
(a) forming the composition of any one of claims 65-134 into a formed shape; and
(b) vulcanizing the formed shape
to provide a vulcanized elastomeric article.

146. The process of claim 145, wherein the vulcanizing is performed at an average temperature of from about 120° C. to about 180° C.

147. The process of claim 145, wherein the vulcanizing is performed at an average temperature of from about 140° C. to about 160° C.

148. The process of any one of claims 145-147, wherein the vulcanized elastomeric article is a tire.

149. The process of claim 148, wherein the tire is a passenger vehicle tire, a light truck tire, a heavy truck or bus tire, a motorcycle tire, an agriculture tire, an earthmover tire, an airplane tire, or a racing tire.

150. The process of any one of claims 145-149, wherein the vulcanized elastomeric article is a component of a tire.

151. The process of claim 150, wherein the component is a bead, a belt, a body ply, an inner liner, a sidewall, an undertread, or a tread.

152. The process of any one of claims 150-157, wherein the vulcanized elastomeric article is a rubber overshoe, a sealing strip, an acoustic panel, an air spring, a bellow, a membrane, a tactile sensor, a crash pad, a hose, a conveyor belt, or a flooring.

153. A lubricant composition comprising a lubricant and the compound of any one of claims 1-63.

154. A combustible fuel composition comprising a combustible fuel and the compound of any one of claims 1-63.

155. A fuel additive composition comprising a fuel additive and the compound of any one of claims 1-63.

156. A process for retreading tires, the process comprising:
(a) applying the composition of any one of claims 64-135 to a tire;
(b) disposing a pre-vulcanized tread around the tire;
(c) disposing a curing envelope around the tire; and
(d) vulcanizing the tire.

157. A kit comprising the composition of any one of claims 64-135 and instructions for using the composition in a vulcanizable elastomeric composition.

158. A kit comprising the composition of any one of claims 64-135 and instructions for using the composition to prepare a vulcanized elastomeric article.
